(19) United States
(12) Patent Application Publication
(10) Pub. No.: US 2014/0335580 A1
(43) Pub. Date: Nov. 13, 2014

(54) Method for Enrichment of Eicosapentaenoic Acid and Docosahexaenoic Acid in Source Oils

(71) Applicant: Board of Supervisors of Lousiana State University and Agricultural and Mechanical College, Baton Rouge, LA (US)

(72) Inventors: Subramaniam Sathivel, Saint Gabriel, LA (US); Huaixia Yin, Cypress, TX (US)

(73) Assignee: Board of Supervisors of Lousiana State University and Agricultural and Mechanical College, Baton Rouge, LA (US)

(21) Appl. No.: 14/345,416

(22) PCT Filed: Sep. 19, 2012
(86) PCT No.: PCT/US12/55983

§ 371 Date: Mar. 18, 2014

Related U.S. Application Data
(60) Provisional application No. 61/537,259, filed on Sep. 21, 2011.

(51) Int. Cl. C12P 7/64

(52) U.S. Cl. CPC C12P 7/6427
USPC 435/134

(57) ABSTRACT

An simpler, cheaper method for enhancing the percentage of eicosapentaenoic acid (EPA) and/or docosahexaenoic acid (DHA) from source oils has been discovered. This method hydrolyzes the oil using the lipase enzyme but does not inactivate the lipase enzyme with either added chemicals or increased temperature. The method relies on centrifugation to separate the enzyme from the desired oil and separate the oil from aqueous impurities. The hydrolyzed oil is enhanced with EPA and DHA, and can be further purified by using activated surface adsorbents, e.g., activated alumina, to remove more free fatty acids and impurities.

The benefit of the filing date of provisional U.S. application Ser. No. 61/537,219, filed 22 Sep. 2011, is claimed under 35 U.S.C. §119(e) in the United States, and is claimed under applicable treaties and conventions in all countries.

This invention was made with government support under USDA/ARS grant number 58-5341-9-429. The Government has certain rights in the invention.

### TECHNICAL FIELD

This invention relates to a method to enhance percentages of eicosapentaenoic acid (EPA) and docosahexaenoic acid (DHA) from various source oils, such that the enriched oil is suitable for human consumption.

### BACKGROUND ART

Importance of Fatty Acids

Lipids provide a concentrated source of energy and essential fatty acids through daily dietary intake. They also serve as important constituents of cell walls and carrier of fat-soluble vitamins. Edible oils are mainly composed of triacylglycerols (also called neutral fats or triglycerides) with phospholipids and glycolipids comprising a small fraction. Triacylglycerols are the combination of one unit of glycerol with three units of fatty acids. A fatty acid contains a long hydrocarbon chain and a terminal carboxylate group. Fatty acids have 3 major physiological roles: (1) Fatty acids are fuel molecules for body metabolism when fatty acids mobilized from triacylglycerols are oxidized to provide energy for a cell or organism, particularly. Fatty acids are the main source of energy when undergoing moderate exercise or resting. (2) Fatty acids are used to modify protein by a covalent bond to target the protein to membrane locations. (3) Derivatives of fatty acid derivatives are used as intercellular (e.g., hormones) and intracellular messengers.

A fatty acid chain may contain from about two to more than thirty carbon atoms which can be linked with single or double bonds. The fatty acids without any double bond linkages are called saturated fatty acids; fatty acids containing one double bond linkage are called mono-unsaturated fatty acids; and fatty acids with more than one double bonds are called polyunsaturated fatty acids (PUFA).

In the chemical nomenclature for unsaturated fatty acids, the position of the double bonds plays an important role. The position of the double bonds can be numbered from the end carboxyl group. However, in the commercial market, as an alternative scheme, the position of the double bonds is numbered from the terminal methyl group, i.e., the carbon furthest from the carboxyl group, which is called the “ω-carbon.” The position of the first double bond from the ω-carbon is notated as ω-x or n-x, where x is the carbon number on which the double bond occurs. For example, an ω-3 PUFA would have the double bond on the third carbon from the end methyl group.

Fish oils, which are rich in long chain ω-3 PUFA, have received attention in the scientific and industrial areas because of their reported positive role in human health. The potential benefits of ω-3 PUFAs in the diet include reduced risk of several diseases, including, cardiovascular diseases, hypertension, atherosclerosis, inflammatory and autoimmune disorders. The polyunsaturated fatty acids in fish oils, especially eicosapentaenoic acid (C20:5n3, EPA) and docosahexaenoic acid (C20:6n3, DHA), have been shown to have a positive effect on preventing a variety of human diseases and disorders (Uauy et al., 2000; Wanasundara et al., 1998; Benatti et al., 2004; Horrocks et al., 1999). The primary source for either EPA or DHA is from extraction from natural fats. The market for oils enriched with ω-3 PUFAs, especially EPA and DHA, is growing with the public awareness of the benefits from these enriched oils. There is a need for better methods to enhance the concentration of EPA and DHA in source oils.

EPA and DHA are primarily found in marine oils; however, unrefined marine oils also contain saturated fatty acids and other impurities such as sterols, waxes, lipid soluble vitamins, phenols and other constituents. Accordingly, marine oils must be purified prior to consumption. In addition, marine oils are known to degrade during processing and in storage. Any enhancement or extraction procedure should be careful to limit degradation to a minimum.

Overconsumption of fish oil or other oils to obtain benefits from ω-3 PUFAs may increase the intake of cholesterol and other saturated fatty acids which could have deleterious health effects (Shahidi et al., 1997). PUFA concentrates containing higher concentrations of EPA and DHA are better at reducing the intake of the undesirable saturated fatty acids than the natural marine oils since the daily intake of total lipid can remain low and, in particular, reduce the intake of more saturated fatty acids (Wanasundara et al., 1998).

Purification Methods for Natural Oils

The impurities found in natural oils and produced from processing and storage of oils may decrease product quality or processing efficiency. These impurities include moisture, dust, protein degradation products, free fatty acids, phosphatides, oxidation products, pigments, trace elements (e.g. copper, iron, sulfur, and halogens), polysaccharides and chlorinated pesticide residues (Young et al., 1994). An objective of purifying natural oils, including fish oil, is to remove any impurities which cause unattractive appearance or are potentially harmful components, while retaining the components known to be beneficial, for example, certain pigments, omega-3 fatty acids, and tocopherols.

Many purification methods have been proposed, usually consisting of a combination of various steps, including without limitation, degumming, neutralization, bleaching, deodorization, and distillation. Fish oils, especially those containing higher concentrations of unsaturated fatty acids, are especially prone to oxidation during these processing, especially if high heat or other extreme conditions are used. Table 1 lists the common purification steps and the typical components removed.

TABLE 1
Purification Method and Components Removed

| PURIFICATION | |
|---|---|
| METHOD | MATERIAL(S) REMOVED |
| Degumming | Phospholipids, trace metals, pigments, carbohydrate, |
| | proteins |
| Neutralization | Free fatty acids, phospholipids, trace metals, pigments, |
| | sulfur, and insoluble matter |
| Bleaching | Pigments, oxidation products, trace metals, traces of |
| Deodorization | soap Free fatty acids, mono- and diacylglycerols, |
| | oxidation products, pigment decomposition |
| Washing | Soap |
| Distillation or | Water |
| other drying | |
| Filtration | Separate bleaching earth |

During the degumming process, phospholipids are removed from natural oils usually using water or acids (Hui, 1996). Neutralization of oils removes free fatty acids and other impurities by adding alkali, usually diluted sodium hydroxide. However, use of alkali may cause both physical and chemical changes in the desired components of the oil. Bleaching removes the color components, usually natural pigments (e.g. carotenoids, chlorophyll, xanthophyll and polyphenols). Bleaching can also remove some oxidation products and suspended mucilaginous and other colloid-like matter (Chang, 1967). Deodorization is a steam distillation process that strips the volatile compounds from the non-volatile oils (Bimbo et al., 1991).

During the above processes for purifying fish oils, thermal degradation and oxidation of polyunsaturated fatty acids (PUFAs) often occur as a result of exposure to high temperatures, solvents and other adverse conditions during processing. Moreover, the conventional purification methods are both labor-intensive and expensive. There is an unmet need for an easier and economical purification method for edible oils that preserves the PUFAs.

Purification by Adsorption:

Adsorption is an alternative method to refine oils. This method involves mass transfer from the fluid phase of an adsorbate that will bind to the adsorbent surface until thermodynamic equilibrium is reached. Adsorption is a cost effective method with less oil loss and less lipid oxidation due to the mild conditions. Different types of adsorbents have been used in purification of edible oils, such as activated earth, activated carbon, kiselguhr, and diatomaceous earth, metal oxide and metal phosphate adsorbents (Bera et al., 2004; Chapman, 1994). Activated earth is by far the most common adsorbent for purification and color improvements of fats and oils (Du et al., 2006; Lara et al., 2004).

Activated earth is a product made by the activation of bentonite (a form of natural clay) using mineral acids under heating for a few hours. Activation results in a strongly protonated clay mineral surface and increased specific surface area from an original 40-60 to about 200 m² per gram of dry clay (Hymore, 1996).

Activated alumina, another adsorbent, is a porous dry powder made by thermal treatment of aluminum hydroxide with a series of non-equilibrium forms of partially hydroxylated aluminum oxide (Al₂O₃). The surface of activated alumina is a complex mixture of aluminum, oxygen, and hydroxyl ions which combine in specific ways to produce both acid and base sites. This increases surface activity and is in adsorption applications (Fleming, 1991).

Chitosan is a product of deactylation of chitin, and is composed of N-acetylglucosamine (GlcNAc) and glucosamine (GlcN) residues. Chitosan is the only natural cationic polysaccharide in nature. Chitin is widely found in the exoskeleton of crustaceans, the cuticles of insects, and the cell walls of fungi. Chitosan has very good adsorption capacity for dyes and metal ions due to the presence of a large number of free amino (—NH₂) groups that can serve as, the coordination and reaction sites (Huang et al., 2010). It has also been reported as reported as a water adsorbing agent (Mucha et al., 2005).

A novel adsorbent from rice hulls has been reported for edible oil processing (Proctor, 1996). Rice hulls, a by-product of rice processing, are rich in amorphous silica, and are very effective in binding phospholipids in adsorption process. In addition, charred sawdust has been used as adsorbent to refine several kinds of edible oils (Bera et al., 2004). Activated carbons have been widely used as adsorbents in technologies related to pollution abatement, pharmaceutical, and food industries due to their highly porous structure, big internal surface area and large adsorption capacity (Song et al., 2005).

Enrichment of ω-3 Polyunsaturated Fatty Acids

The common techniques used to concentrate PUFAs include urea fractionation, low temperature fractional crystallization, salt solubility methods, gas chromatography, and thin-layer chromatography. These techniques usually fractionate the fatty acids based on the number of double bonds or the chain length. However, not all those methods are applicable to a large production scale (Robles et al., 1998). In addition, recent processes to concentrate PUFAs include supercritical fluid technology and lipase-catalyzed hydrolysis reaction.

EPA was purified by urea fractionation and distillation processes, but some degree of cis-trans conversion was reported, a result undesirable for food or pharmaceutical use (see U.S. Pat. No. 4,377,526). Low temperature fractional crystallization, another commonly used method, is usually carried out in organic solvents, such as acetone (Shinowara et al., 1940). The salt solubility method uses lithium soaps dissolved in acetone and alcohol for separation since the lithium salts of polyenoic fatty acids are soluble in 95% acetone while less unsaturated acids are relatively insoluble (Marldey, 1964). Supercritical fluid technology was used to separate a mixture of fatty acid ethyl esters but the cost was very high (Eisenbach, 1984). Other methods such as thin-layer chromatographic and gas chromatographic methods require undesirably high amounts of organic solvents.

Most of the methods described above produce a PUFA concentrate with the form of the PUFAs as their corresponding alkyl esters. Several studies revealed that alkyl esters of ω-3 fatty acids can impair intestinal absorption in laboratory animals (El-Boustani et al., 1987; Hamazaki et al., 1982; Lawson et al., 1988). The acylglycerol form of a PUFA is considered to be nutritionally more favorable than a methyl or ethyl esters.

Thus, a variety of methods have been used to enrich marine oils with EPA and DHA. Many of these methods require extreme physical and chemical conditions and cause some degree of degradation of the fatty acids, formation of peroxides, and conversion of some of the cis-bonds to the trans-form, another undesirable outcome. Furthermore, many of the materials added during the enrichment process, such as acetone, hexane, and other organic solvents, are not on the Generally Recognized as Safe (GRAS) list of the U.S. Food and Drug Administration. These materials would have to be removed from the final product.

Lipase-Catalyzed Hydrolysis Enrichment:

Lipases are important enzymes that specifically hydrolyze carboxyl esters of triglycerides into free fatty acids and partial acylglycerols. The main advantage of lipase hydrolysis compared with other chemical methods is the avoidance of the formation of undesirable oxidation products, polymers, and isomeric conversion of natural cis-PUFAs to deleterious trans-PUFAs. Another important characteristic that lipases offer is selectively (i.e., substrate, positional, and stereospecificity) concentrating on targeted fatty acids in triglycerides (Jaeger et al., 1998). EPA and DHA can be concentrated by the lipase-assisted hydrolysis because the 5 or 6 double bonds found in EPA and DHA result in molecules that are bent (i.e., not linear), so that the molecule lies close to the ester bond and the lipase is less likely to hydrolyze the EPA and DHA ester bond (Bottino et al., 1967).

Several microbial lipases have been used to produce ω-3 PUFA concentrates in the form of acylglycerols by hydrolysis of marine oils (Tanaka et al., 1992; Hoshino et al., 1990; Shimada et al., 1994; Yadwad et al., 1991; Maehr et al., 1994). Lipase-catalyzed enzymatic production of EPA and DHA concentrate from fish oil has been reported to have potential in producing a high quality product because of the mild conditions of the process (Breivik et al., 1997). Table 2 summarizes the properties of several of these lipases and their effectiveness on different oils.

TABLE 2
Effectiveness of lipase on EPA and DHA enrichment of marine oils

| | Literature | | | Lipase |
|---|---|---|---|---|
| Lipase source | Source(s) | Oil | Effectiveness | properties |
| Aspergillus niger | Sun et al., | Atlanta | Ineffective in increasing EPA | Origin: Fungal |
| | (2002) | salmon oil | and DHA | Optimal |
| | Wanasundara | Seal blubber | Ineffective in hydrolyzing | temperature and |
| | et al. (1998) | oil and | saturated fatty acids | pH: 30-40° C., 6.5 |
| | | menhaden oil | | Positional |
| | Okada et al., | Sardine oil | No increase in EPA and minor | specificity: 1, 3- |
| | (2007) | | increase in DHA | specific |
| Pseudomonas | Sun et al., | Atlanta | Ineffective in concentrating | Origin: Bacterial |
| fluorescens | (2002) | salmon oil | EPA and DHA | Optimal |
| | | | | temperature and |
| | | | | pH 45-55° C.; 8.0 |
| | | | | Positional |
| | | | | specificity: none- |
| | | | | specific |
| Candida rugosa | Sun et al., | Atlanta | Increased 42% EPA at 12 h, | Origin: Yeast |
| | (2002) | salmon oil | increased 72% DHA at 12 h | Optimal |
| | Okada et al., | Sardine oil | Increased EPA from 26.87% to | temperature and |
| | (2007) | | 33.74%, DHA from 13.62% to | pH: 30-50° C.; 7.0 |
| | | | 29.94%. | Positional |
| | | | | specificity: none- |
| | | | | specific |
| Rhizopus oryzae | Sun et al., | Atlanta | Ineffective in concentrating | Origin: Fungal |
| | (2002) | salmon oil | EPA and DHA | Optimal |
| | Wanasundara | Seal blubber | Increased DHA, decreased EPA | temperature and |
| | et al., (1998) | oil and | | pH: 30-45° C., 7.0 |
| | | menhaden oil | | Positional |
| | | | | specificity: 1, 3- |
| | | | | specific |
| Mucor javanicus | Sun et al., | Atlanta | Ineffective in concentrating | Origin: Fungal |
| | (2002) | salmon oil | EPA and DHA | Optimal |
| | Okada et al., | Sardine oil | No increase in EPA and minor | temperature and |
| | (2007) | | increase in DHA | pH: 30-45° C., 7.0 |
| | | | | Positional |
| | | | | specificity: 1, 3- |
| | | | | specific |
| Pseudomonas | Sun et al., | Atlanta | Increased 60% EPA at 12 h, | Origin: Bacterial |
| cepacia | (2002) | salmon oil | increased 58% DHA at 12 h | Optimal |
| | | | | temperature and |
| | | | | pH: 30-65° C., 7.0 |
| | | | | Positional |
| | | | | specificity: none- |
| | | | | specific |
| Candida | Okada et al., | Sardine oil | Increased EPA and DHA | Optimal |
| cylindracea | (2007) | | contents | temperature and |
| | | | | pH: 30-50° C., 6.5 |
| | | | | Positional |
| | | | | specificity: none- |
| | | | | specific |
| Mucor miehei | Wanasundara | Seal blubber | Ineffective in hydrolyzing | Optimal |
| | et al., (1998) | oil and | saturated fatty acids in both oils | temperature and |
| | | menhaden oil | | pH: 30-45° C., 6.5-7.5 |
| | | | | Positional |
| | | | | specificity: 1, 3- |
| | | | | specific |
| Rhizopus niveus | Wanasundara | Seal blubber | Increased 25.3% n-3 fatty acids | Optimal |
| | et al., (1998) | oil and | in seal blubber oil, increased | temperature and |
| | | menhaden oil | 23% n-3 fatty acids in | pH: 30-45° C., 5.0-8.0 |
| | | | menhaden oil | Positional |
| | | | | specificity: 1, 3- |
| | | | | specific |
| Candida | Wanasundara | Seal blubber | Increased 50% EPA and 5 fold | Optimal |
| cylindracea | et al., (1998) | oil and | of DHA in seal blubber oil; | temperature and |
| | | menhaden oil | increased 60% EPA and 70% | pH: 30-50° C., 5.0-8.0 |
| | | | DHA in menhaden oil | Positional |
| | | | | specificity: none- |
| | | | | specific |
| Chromobacterium | Wanasundara | Seal blubber | Increased 54% EPA and | Positional |
| viscosum | et al., (1998) | oil and | ineffective to DHA in seal | specificity: none- |
| | | menhaden oil | blubber oil; increased 50% EPA | specific |
| | | | and ineffective to DHA in | |
| | | | menhaden oil | |
| Geotrichum | Wanasundara | Seal blubber | Increased 46% EPA and 2.5 | Origin: Fungal |
| candidum | et al., (1998) | oil and | fold of DHA in seal blubber oil; | Optimal |
| | | menhaden oil | increased 38% EPA and 50% | temperature and |
| | | | DHA in menhaden oil | pH: 30-45° C., 6.0-8.0 |
| | | | | Positional |
| | | | | specificity: none- |
| | | | | specific |
| Pseudomonas sp. | Wanasundara | Seal blubber | Increased 64% EPA and | Origin: Fungal |
| | et al., (1998) | oil and | ineffective to DHA in seal | Optimal |
| | | menhaden oil | blubber oil; increased 50% EPA | temperature and |
| | | | and ineffective to DHA in | pH: 40-60° C., 5.0-9.0 |
| | | | menhaden oil | Positional |
| | | | | specificity: none- |
| | | | | specific |
| Aspergillus oryzae | Matouba et al., | Salmon Oil | Not very effective (increased | Optimal |
| | (2008) | | 16.8% of EPA + DHA) | temperature: |
| | | | | 37° C. |
| | | | | Positional pH: 7 |
| | | | | Specificity: 1, 3- |
| | | | | specific |

Many studies have been conducted to enrich fish oil with EPA and DHA in the form of glycerol (Sun et al., 2002; Wanasundara et al., 1998; Linder et al., 2005). In most of the methods, fish oils were hydrolyzed with lipase, then the lipase subsequently chemically inactivated, and the free fatty acids (FFA) generated were neutralized with KOH or NaOH, followed by enriched oil separation by hexane. One published method to produce PUFAs from marine oils using eight different microbial lipases is shown in FIG. 1 (Wanasundara et al., 1998). As shown in FIG. 1, this method consisted of five steps: hydrolysis of triglycerides, inactivation of lipase, neutralization of free fatty acids, separation of n-3 PUFAs with hexane, and evaporation of hexane.

In methods using lipase, lipase activity is usually stopped by solvents, such as methanol (Wanasundara et al., 1998), ethanol (Okada et al., 2007), and acetone:ethanol mixture (Liu et al., 2007). These solvents were then evaporated to be removed from the oil. This addition of solvents increases the cost and adds the risk of negative health effects. Alternatively, thermal treatment is a conventional method to inactivate enzyme. Temperature has been used to inactivate the lipase, but has a negative effect on the quality of the oil by oxidizing it. Filtration has also been used to remove the immobilized lipases to discontinue the hydrolysis reaction (Breivik et al., 1997).

After inactivation of the enzyme, FFAs are usually neutralized by KOH or NaOH solution (Wanasundara et al., 1998; Okada et al., 2007), forming a soap (“saponification”). Multiple additions of hexane and water are then needed to extract the oil and remove the soap. These additions may actually accelerate the formation of FFAs, and hexane could be a negative compound present in oil even after evaporation.

Disadvantages with the enhancement methods currently used are several. A high volume of added organic solvents and chemicals are used to produce the oil enriched with EPA and DHA, increasing the costs. Many of these organic solvents and chemicals cause negative health effects, and thus must be removed from the final product. Additionally, most enrichment methods increase the PUFAs that are in the form of free fatty acids or their corresponding alkyl esters. There is a strict limit for the content of FFA in fish oils for human consumption. There are differences in how the alkyl, methyl and ethyl esters are handled by the organism. Methyl and ethyl esters of unsaturated fatty acids are reported to hydrolyze at a slower rate than their corresponding acylglycerols (Yang et al., 1989). Several studies revealed the fact that alkyl esters of n-3 fatty acids can impair intestinal absorption in laboratory animals (El-Boustani et al., 1987; Hamazaki et al., 1982; Lawson et al., 1988). The acylglycerol form of PUFA is considered to be nutritionally more favorable than methyl or ethyl esters of fatty acids.

### DISCLOSURE OF INVENTION

We have discovered a new method to process oils to enhance and purify the oil so that the subsequent product has a higher concentration of EPA and DHA. This new method is cheaper and preserves EPA and DHA greater as compared to conventional methods. This method uses enzymatic (lipase) hydrolysis for enriching oils with EPA and DHA in form of acylglycerols, but our method does not add chemicals to inactivate the enzyme. In addition, the method does not need to add a base to neutralize free fatty acids. In a preferred embodiment, a two-step protocol was used to first produce enriched fish oils and then to further purify the enriched oil, preferably using adsorption techniques. Such a combined, continuous two-step method (EPA and DHA enrichment followed by activated surface purification) was shown to produce purified oils enriched with EPA and DHA. In one embodiment of the invention, the chosen lipase was isolated from Candida rugosa.

Prior to the present invention, enzymatic hydrolysis of oils was followed by enzyme inactivation either chemically or thermally. This inactivation/denaturation was done prior to the enzyme removal from its lipid substrate by centrifugation. Set forth herein is a method for enhancing the percentage of EPA or DHA in oil without the need for chemical or thermal enzyme inactivation of the particulate lipase enzyme. Our method for enhancing EPA and DHA comprises (a) hydrolyzing a source oil containing EPA or DHA with a particulate lipase that hydrolyzes the a carboxyl ester of triglycerides into free fatty acids and acylglycerols; (b) separating the hydrolyzed oil into layers based on hydrophobicity by subjecting the hydrolyzed oil to centrifugation (three layers are formed during centrifugation: an aqueous phase bottom layer, a middle layer comprising particulate lipase, and an upper oil layer comprising enhanced amounts of EPA or DHA along with free fatty acids); and (c) collecting the enhanced oil from the oil layer. Preferably, the collected oil is further exposed to an activated surface material. The collected oil comprises free fatty acids and impurities, of which at least some are retained by the activated surface material. The activated surface material is then removed, leaving behind an oil with an enhanced percentage of EPA or DHA and decreased amounts of free fatty acids and impurities. The original source oil can be any source known to contain EPA and/or DHA, for example, fish oils or marine mammalian oils. Examples of fish oils that have high levels of EPA and/or DHA are menhaden (Brevoortia species), sardine and salmon (species of Salmo and Onchorhynchus) oils. A good example of mammalian oil with EPA and/or DHA is blubber from marine mammals, including seals and whales.

The method we have discovered does not have a step of chemical inactivation of the lipase following the hydrolyzing step nor does it have a step of thermal inactivation of the lipase following the hydrolyzing step. The lipase is not inactivated prior to the separation by centrifugation. In a preferred embodiment, our method does not have a step of neutralization using an alkali following the lipase hydrolyzing step, and does not have the step of distillation following the hydrolyzing step. In accordance with the invention, the lipase is chosen based on the ability to hydrolyze other fatty acids, but not specifically or at least minimally hydrolyze DHA and/or EPA. Examples of such lipases can include, lipase produced by an organism selected from the group consisting of Candida rugosa, Pseudomonas cepacia, Candida cylindracea, Rhizopus niveus, Chromobacterium viscosum, Geotrichum candidum, Pseudomonas sp., and Aspergillus oryzae. In certain preferred embodiments, the lipase is produced by Candida rugosa. A preferred purification method for the oil collected from centrifugation is use of activated surface materials, including without limitations, activated alumina, activated earth and chitosan. The collected oil may be exposed to activated surface material one or more times to bind and remove free fatty acids and other impurities, with removing the activated surface material following each exposure. The surface activated material can be removed from the oil in various methods, including centrifugation. Table 3 gives a list of common abbreviations used herein.

TABLE 3
List of Common Abbreviations

| Abbreviation | Abbreviated Term |
|---|---|
| CR | Candida rugosa |
| DG | Diglyceride |
| DH | Degree of hydrolysis |
| DHA | Docosahexaenoic acid (C23:6n3) |
| EPA | Eicosapentaenoic acid (C20:5n3) |
| FAME | Fatty acid methyl ester |
| FFA | Free fatty acid |
| IV | Iodine value |
| MG | Monoglyceride |
| MO | Menhaden fish oil |
| MOE | Menhaden oil enhanced |
| PUFA | Polyunsaturated fatty acid |
| PV | Peroxide value |
| SO | Salmon oil |
| SOE | Salmon oil enhanced |
| TBA | Thiobarbituric acid |
| TBAR | Thiobarbituric acid-reactive substance |
| TG | Triglyceride |

### BRIEF DESCRIPTION OF THE DRAWINGS

FIG. 1 is a schematic showing a common process for enzymatic hydrolysis of marine oils and separation of acylglycerols and free fatty acids (Wanasundara et al., 1998).

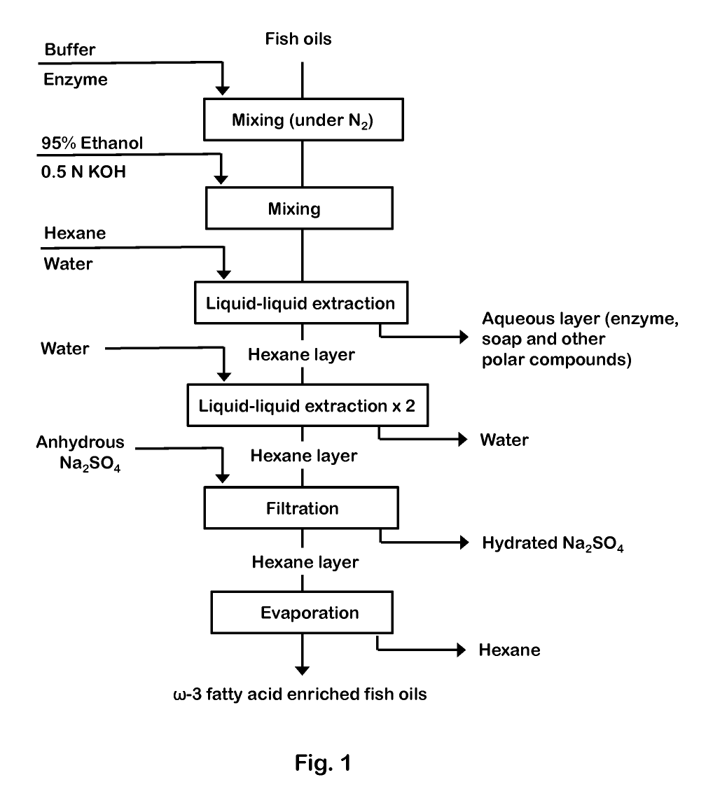

FIG. 2 is a schematic showing the new, simpler method for enzymatic hydrolysis of source oils and further purification using activated surface adsorbent.

FIGS. 3A-3E show changes in the concentrations of certain hydrocarbons—C16:0 (FIG. 3A), C16:1n7 (FIG. 3B), EPA (FIG. 3C), DHA (FIG. 3D), and EPA+DHA (FIG. 3E)—in menhaden oils hydrolyzed for up to 24 h with three different lipase amounts (250 U, 500 U, and 2500 U).

FIGS. 4A-4F show changes in the concentrations of certain hydrocarbons—C16:0 (FIG. 4A), C16:1n7 (FIG. 4B), C18:1n9 (FIG. 4C), EPA (FIG. 4D), DHA (FIG. 4E), and EPA+DHA (FIG. 4F)—in salmon oils hydrolyzed for up to 24 h with three different lipase amounts (50 U, 250 U, and 1250 U).

FIGS. 5A-5C show changes in triglycerides (TG), diglycerides (DG), and monoglycerides (MG) fractions of menhaden oils hydrolyzed for up to 24 h at three different lipase amounts—250 U (FIG. 5A), 500 U (FIG. 5B), and 2500 U (FIG. 5C).

FIGS. 6A-6C show changes in triglycerides (TG), diglycerides (DG), and monoglycerides (MG) fractions of salmon oils hydrolyzed for up to 24 h at three different lipase amounts—50 U (FIG. 5A), 250 U (FIG. 5B), and 1250 U (FIG. 5C).

### MODES FOR CARRYING OUT THE INVENTION

Set forth herein is an enzyme-based enrichment method for EPA and/or DHA that minimizes the degradation and oxidation of PUFAs. Optionally, the enriched oil is then purified using an activated surface adsorbent. In the enrichment method and optional purification, the use of costly and potentially unhealthy chemicals is avoided. Moreover, the enrichment method avoids the use of excessive temperatures to inactivate the lipase enzyme, but also degrades the quality of the enhanced oil.

Menhaden (MO) and salmon (SO) oils were used to develop and to confirm the efficacy of this new method. MO and SO were hydrolyzed by Candida rugosa lipase at enzyme concentrations of 250, 500, 2500 Units (U) per 50 g MO and 50, 250, 1250 Upper 50 g SO, for up to 24 h. By using centrifugation, the enzyme and the oil were separated, thus removing the enzyme from further interaction with fatty acid substrates in the oil. The enzyme was removed using centrifugal force. The fish oils enriched with EPA and DHA were collected from the centrifuged sample (the top layer), and the enriched oil further purified using adsorption technology.

As shown in the examples herein, total EPA and DHA fractions increased from 21.1% to 38.9% for MO at 2500 U lipase and from 20.1% to 32.8% for SO at 1250 U lipase after 6 h of hydrolysis. In addition, the activated surface adsorption process reduced the impurities in fish oil that had been enriched with EPA and DHA.

For the data that follows, unrefined menhaden fish oil (MO) and unrefined salmon oil (SO) was used, with the microbial lipase from Candida rugosa. The menhaden oils were treated with 50, 250, and 1250 U of lipase for 3, 6, 12, and 24 h, while salmon oils were hydrolyzed with 250, 500, and 2500 U of lipase for 3, 6, 12, and 24 h. The degree of hydrolysis (DH) and acylglycerol fractions (monoglycerol (MG), diglycerol (DG), and triglycerol (TG)) were determined after the hydrolysis reaction. The final oils were collected for fatty acid composition analysis.

The enzyme amount and hydrolysis time during the process were optimized with regards to high EPA and DHA production. Menhaden and salmon purified oil enriched with EPA and DHA were produced, and then analyzed for the fatty acid composition, perioxidase value (PV), FFA, thiobarbituric acid-reactive substance (TBARs), rheological properties, and color. As shown below, our method produced fish oils enhanced with EPA and DHA, and with few impurities.

### Example 1

### Materials and Methods

Sample preparation: Unrefined Gulf menhaden (Brevoortia patronus) fish oil (MO) extracted using a rendering process was obtained from a commercial source (Omega Protein Inc., Houston, Tex.). Unrefined salmon oil (SO) was produced from processing salmon byproducts including viscera, heads, skins, frame, and discarded fish obtained from a large commercial plant in Alaska. The salmon oil was from a combination of two species: red salmon (Oncorhynchus nerka) and Pink salmon (Oncorhynchus gorbuscha). The microbial lipase (Candida rugosa, CR) was purchased from Sigma-Aldrich Co., St. Louis, Mo.

Determination of Enzyme Activity:

A commercially available microbial lipase (Candida rugosa, CR) was selected to hydrolyze the fish oils. CR lipase is extracted from yeast and is non-specific to the fatty acid positions on triglycerides. The optimum temperature and pH are 30-50° C. and 7.0. Enzyme activity of the lipase was determined by the enzymatic assay of lipase (EC3.1.1.3) from Sigma-Aldrich substituting menhaden or salmon oil for olive oil. Free fatty acids (FFA) released by the hydrolysis reaction (30 min) were titrated against 0.5 N sodium hydroxide and the pH changes were monitored by adding 0.1 mL thymolphthalein indicator solution (0.9% w/v). One unit of enzyme activity (U) was defined as the amount of enzyme that liberated 1 μmol of fatty acid in 1 h at 37° C.

Hydrolysis and Separation:

The hydrolysis of fish oils by CR lipase, and the separation of the EPA and DHA enriched fraction were carried out by the following procedure. Enzyme powder representing different amounts of lipase was dissolved in 25 ml of phosphate buffer, pH 7.0, and then mixed with 50 g of MO or SO in an amber bottle. The air in the bottle was replaced by N₂, and the bottle was capped to minimize lipid oxidation. The hydrolysis reaction was maintained at 37° C. in an incubator shaker (model 3525, LAB-LINE Instruments Inc., Melrose park, Ill.) at 250 rmp for the desired time for hydrolysis (from about 1 to about 24 hr). Then the lipase/oil mixture was separated by centrifugation at 10,000 rpm for 10 min at 10° C. with a Beckman J2-HC (GMI Inc., Ramsey, Minn.). It is understood by those of ordinary skill in the art that centrifugation time and temperature may change depending on the type of centrifuge, and the melting point of the relevant oil.

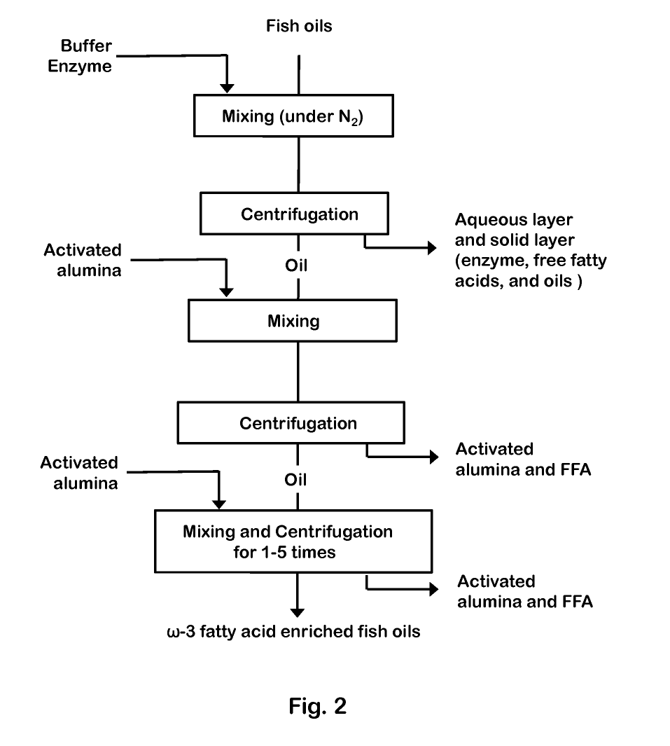

Three layers were formed in the centrifuge tubes after centrifugation. The bottom layer was the aqueous phase. The middle layer contained the enzyme powder and a small amount of fish oil or impurities in the fish oil. The upper layer was the desirable, hydrolyzed fish oil with high amounts of free fatty acid. The top layer of fish oil was collected and based on the data on oil purification (described below), activated alumina (AA) was then used to adsorb the FFA from the fish oils. Sixty percent (w/w) AA was added to the fish oil and agitated with a magnetic stir bar at 60° C. for 1 h. The AA, which was saturated with free fatty acids, was then separated from the oil by centrifugation at 12,000 rpm for 10 min at 4° C. This step was repeated until all the FFA was removed from the oil. FIG. 2 is a schematic showing the above process. As indicated in FIG. 2, the addition of activated alumina followed by centrifugation is a step that may be repeated from one to five or more times. Final EPA and DHA enriched menhaden (MOE) or salmon oil (SOE) was flushed with N₂ and stored at −20° C. until further use.

Determination of Degree of Hydrolysis (DH) in the Hydrolyzed Menhaden and Salmon Oils:

Degree of hydrolysis (DH) was determined by measuring the acid value of both raw and hydrolyzed oil as well as saponification value of raw oil according to American Oil Chemists' Society (AOCS) methods (AOCS 1998). Blanks (no enzyme) were determined at each treatment. DH was calculated according to the following equation:

$$DH ( \% ) = \frac{acid value ( hydrolyzed oil ) - acid value ( raw oil )}{saponification value ( raw oil ) - acid value ( raw oil )} \times 100$$

In the above equation, acid value is expressed as the number of mg of KOH required to neutralize free fatty acids present in 1 g of oil; the saponification value is defined as the number of mg of KOH required to saponify 1 g of oil.

Analysis of Acylglycerol Composition of Hydrolyzed Menhaden and Salmon Oils:

Acylglycerol composition was analyzed at the laboratory of the W. A. Callegari, Environmental Center, Louisiana State University, La., following the ASTM international standard method D-6584 with minor modification. A 20 mg sample was weighed into a 10 mL septa vial. Using microlitre syringes, exactly 100 μL of each internal standard and MSTFA were added. After shaking the vials, they were allowed to sit for 15 to 20 min at room temperature. Approximately 2 mL hexane was added to the vial and shaken. One μL of the reaction mixture was injected into the cool on-column injection port of the gas chromatography. The initial temperature was 50° C. for 1 min, then the temperature was increased to 180° C. at a rate of 15° C./min, and then 7° C./min to 230° C., finally 30° C./min to 380° C. and held for 10 min. Helium was used as the carrier gas with a flow rate of 3 mL/min and a flame ionization detector was used to detect the peaks. The peaks were identified by comparing retention times with the standards. The mono-, di, and triglycerides were separated according to carbon numbers (CN). Monoglycerides consist of the four overlapping peaks with relative retention times (RRT) of 0.76 and 0.83 to 0.86 with respect to the internal standard tricaprin. The grouping of 3 to 4 peaks with RRT of 1.05 to 1.09 (CN 34, 36, and 38) was attributed to diglycerides. Peaks with RRT of 1.16 to 1.31 (CN 52, 54, 56, and 58) were included in the calculation.

Changes During the Enrichment of EPA and DHA in Menhaden and Salmon Oils:

The menhaden and salmon oils were hydrolyzed with lipase at different units (50, 250, 500, 1250, 2500, and 5000 U) for 1 h. The FFA and DH were determined after each hydrolysis treatment. The menhaden, oils were treated according to the procedure described by FIG. 2 with 250, 500, and 2500 U of lipase for 3, 6, 12, and 24 h, while salmon oils were hydrolyzed with 50, 250, and 1250 U of lipase for 3, 6, 12, and 24 h. The DH and acylglycerol fractions (MG, DG, and TG) were determined after the hydrolysis reaction. The final oils were collected for fatty acid composition analysis.

The enzyme amount and hydrolysis time during the process were optimized with regards to high EPA and DHA production. Menhaden and salmon enriched with EPA and DHA were produced at the optimum conditions and analyzed for the fatty acid composition, PV, FFA, TBARs, rheological properties, and color.

Fatty Acid Methyl Ester (FAMEs) Composition of the Fish Oils:

Fatty Acid composition of oil, samples was determined at the USDA-ARS Laboratory, University of Alaska Fairbanks, Ak. FAMEs were prepared using a modified method (Maxwell et al., 1983). A 20 mg sample of fish oil was dissolved in 4.5 mL isooctane and 500 μL of internal standard (10 mg methyl tricosanoate (23:0)/mL isooctane) and 500 μL 2 N KOH (1.12 g/10 mL MeOH) was added to the mixture. The mixture was vortexed for 1 min and centrifuged to the separate upper layer. The separated upper layer was mixed with 1 mL of saturated ammonium acetate solution and the aqueous layer was removed and discarded. The mixture was centrifuged and the upper layer of the mixture was separated. Then 1 mL of distilled water was added to the separated upper layer and centrifuged, then 2-3 g anhydrous sodium sulfate was added, vortexed, and kept for 20-30 min. The mixture was centrifuged and the liquid containing methyl ester was separated. A 0.5 mL aliquot of isooctane containing methyl ester and 0.5 mL of isooctane were added to the amber GC vial. The fatty acid analysis was done with a GC model 7890A (Agilent) fitted with a FAMEWAX™ (30 m, 0.32 mm×0.25 μm, Restek, Bellefonte, Pa.) column. Data was collected and analyzed using the GC ChemStation program (ver E.02.00.493 Agilent Technologies, Inc.). Helium was used as the carrier gas at an average velocity of 64 cm/sec. Injector and detector temperature were held at 250° C. and 280° C., respectively. A split injection (50:1 split ratio) was used and the oven programming was 195° C. to 240° C. at a rate of 5° C./min and held 2 min for a total run time of 11 min. An autosampler performed the GC injection of standards and samples. The injection volume was 1 μL. Samples were identified by comparing retention times to standards. The standards used were: Supelco 37, PUFA #1, PUFA #3, and cod liver oil from Supelco (Bellefonte, Pa.). Data were expressed as percent of total integrated area.

PV, FFA, TBARs, and Color of the Raw Fish Oil and Final Oil Enriched with EPA and DHA:

Peroxidase value (PV) of the fish oils was determined by a titration method according to AOAC 965.33 (1999). The results were expressed in terms of milliequivalent peroxides per Kg of oil (meq/Kg). FFA content of the unrefined oil was also determined using a titration method (AOCS Ca 5a-40, 1998), and the percentage FFA was expressed as oleic acid equivalents.

A modification of a previously described method (Mei et al., 1998) was employed for measuring the TBARs of oil samples. A thiobarbituric acid (TBA) solution was prepared by mixing 15 g of trichloroacetic acid, 0.375 g of TBA, 1.76 mL of 12 N HCl, and 82.9 mL of H2O. TBA solution (100 mL) was mixed with 3 mL of 2% butylated hydroxytoluene in ethanol, and 2 mL of this solution was mixed with 6 mg of oil sample. The mixture was vortexed for 10 sec and heated in a boiling water bath for 15 min. After the mixture cooled down to room temperature, it was centrifuged at 3400×g for 25 min. The absorbance of the supernatant was measured at 532 nm. Concentrations of TBARS were determined from standard curves prepared with 0-0.02 mmol/L 1,1,3,3-tetraethoxypropane.

Statistical Analysis:

Analysis of Variance (ANOVA) was conducted to evaluate the significance of observed differences among treatment means (SAS version 8.2, SAS Institute Inc., Cary, N.C.), followed by the post-hoc Tukey's studentized range test (SAS 2002).

### Example 2

### Enrichment of EPA and DHA in Menhaden Fish Oil

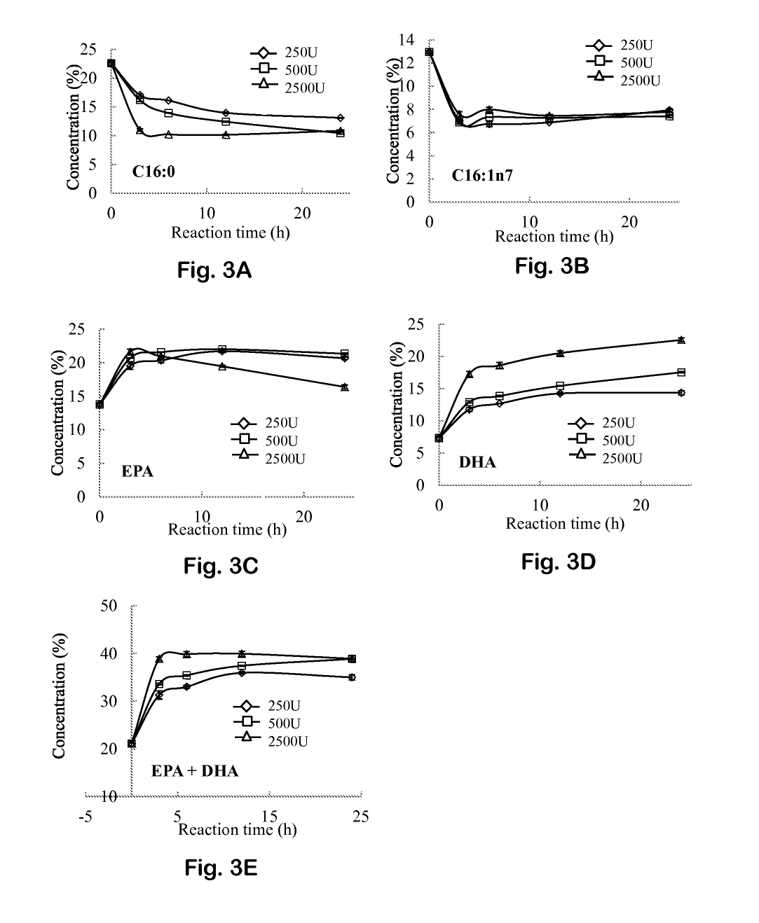

Using menhaden oil, the above process was shown to change the amounts of the following fatty acids: C16:0, C16:1n7, EPA, DHA and EPA+DHA (see FIGS. 3A-3E). As shown in FIGS. 3A-3E, the changes depended on the amount of the added lipase (250 U, 500 U, or 2500 U), and on the time of hydrolysis (up to 24 h). Different amounts of lipase caused changes in fatty acid composition in the menhaden oil.

The C16:0 content decreased significantly (p>0.05) from 22.58% to 17.05% after 3 h and gradually decreased to 13.09% after 24 h with 250 U lipase; the same pattern was observed with lipase at 500 U and 2500 U, except that C16:0 presented at slightly lower levels, ranging from 22.58% to 10.44% and 22.58% to 10.84% (FIG. 3A). Also, levels of C16:1n7 decreased significantly after 3 h from 12.97% to 7.02%, 6.88%, and 7.60% with 250 U, 500 U and 2500 U, respectively; however the levels remained relatively constant for the rest of the hydrolysis reaction time (up to 24 h) (FIG. 3B).

With 250 U CR lipase, EPA content significantly increased from 13.77% to 19.52% after 3 h and remained at relatively constant levels after 6 and 12 h (20.34% and 21.70%, respectively), but after 24 h of hydrolysis, the EPA content decreased to 20.66% (FIG. 3C). This indicates that the lipase started to facilitate hydrolysis of EPA at 24 h with 250 U lipase. A similar tendency was observed with 500 U lipase, except that EPA was present at slightly higher levels, ranging from 13.77% to 22.01%. When 2500 U lipase was applied, EPA levels reached the highest amount at 3 h to 21.64%, and dropped thereafter to 16.37% at 24 h. This result indicated that a high amount of EPA was removed by the hydrolysis reaction at longer time periods.

As shown in FIG. 3D, DHA levels also increased significantly after 3 h, from an original of 7.32% to 11.76% with 250 U lipase, to 12.90% with 500 U lipase, and to 17.26% with 2500 U lipase. Gradual increases in DHA concentration from 11.76% to 14.34% with 250 U, 12.90% to 17.53% with 500 U, and 17.26 to 22.55% with 2500 U occurred as hydrolysis continued to 24 h. A significant increase in DHA was found among 3 h and longer reaction times. The highest total EPA and DHA fraction (39.54% and 39.95%) in the menhaden oil was found in the oil hydrolyzed with 2500 U lipase for 6 and 12 h (FIG. 3E).

### Example 3

### Enrichment of EPA and DHA in Salmon Fish Oil

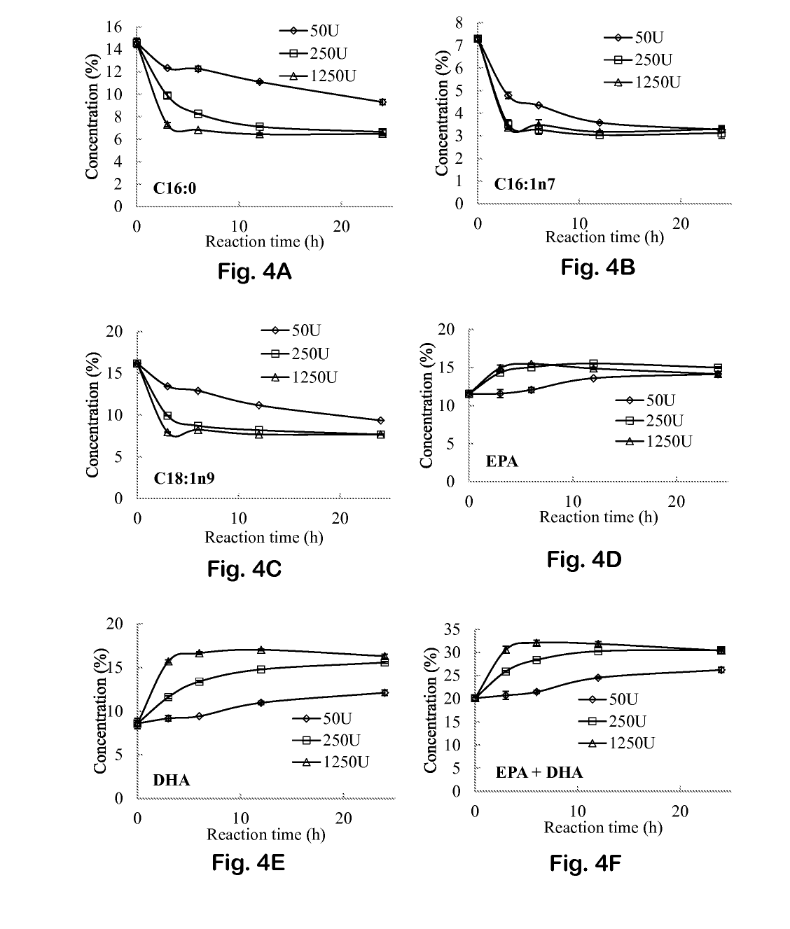

Using salmon oil, the above process as described in Example 1 was shown to change the amounts of the following fatty acids: C16:0, C16:1n7, EPA, DHA and EPA+DHA (sec FIGS. 4A-4E). As shown in FIGS. 4A-4E, the changes depended on the amount of the added lipase (50 U, 250 U, or 1250 U), and on the time of hydrolysis (up to 24 h). Different amounts of lipase caused changes in fatty acid composition in the salmon oil.

As shown in FIG. 4A, the C16:0 content decreased significantly (p>0.05) from 14.58% to 12.34% after 3 h, and gradually decreased to 9.30% after 24 h with 50 U lipase. The same pattern was observed with lipase at 250 and 1250 U, except that C16:0 was present at much lower levels, ranging from 14.58% to 6.64% and 14.58% to 6.47%, respectively. Also, levels of C16:1n7 decreased significantly after 3 h from 7.30% to 4.78%, 3.50%, and 3.76% with 250 U, 500 U and 2500 U, respectively; and then the levels gradually decreased to 3.27%, 3.12% and 3.18%, respectively, with reaction time up to 24 h (FIG. 4B).

As shown in FIG. 4C, the C18:1n9 content decreased significantly from 16.15% to 13.43% after 3 h and gradually decreased to 9.34% after 24 h with 50 U lipase. The same pattern was observed with lipase at 250 and 1250 U, except that it was present at much lower levels, ranging from 16.15% to 7.66% and 16.15% to 7.67%, respectively.

With 50 U lipase, EPA content increased from 11.55% to 14.15% gradually after 24 h (FIG. 4D). With 250 U, EPA content significantly increased from 11.55% to 14.32% after 3 h, and remained at relatively constant levels after 6 and 12 h (15.04% and 15.54%, respectively), but decreased after 24 h of hydrolysis to about 14.99% (FIG. 4D). This indicates the lipase started to facilitate hydrolysis of EPA at 24 h with 250 U lipase. A similar tendency was observed for EPA with 1250 U lipase except that the highest content (15.49%) was achieved at 6 h of hydrolysis and decreased to 14.14% at 24 h of hydrolysis.

As shown in FIG. 4E, DHA content increased gradually from 8.59% to 12.10% after 24 h with lipase 50 U. With lipase at 250 U and 1250 U, DHA levels of the salmon oil increased significantly after 3 h, from an original of 8.59% to 11.59% and 8.59% to 15.68%, respectively. With 250 U lipase, a gradual increase in DHA concentration from 11.59% to 15.56% was seen up to 24 h. However, with 1250 U, the highest level (17.01%) of DHA occurred at 12 h hydrolysis, and then dropped to 16.28% after 24 h of hydrolysis. Again, this indicates that the lipase was hydrolyzing the DHA from the acylglycerol at 24 h from salmon oil with 1250 U lipase. The highest total EPA and DHA fraction (32.12%) in the salmon oil was found in the oil hydrolyzed with 1250 U lipase for 6 h (FIG. 4F).

From the above results, using this method, we found that it was easier to hydrolyze EPA than DHA for the CR lipase in both menhaden and salmon oils. Without wishing to be bound by this theory, we believe this is because EPA (C20:5n3) has less carbons and a shorter chain than DHA (C22:5n3), and thus an easier access for the lipase to the ester bond. Most lipases, including from Candida rugosa, have been found to discriminate against DHA more than EPA (Mukherjee et al., 1993).

### Example 4

### Changes in Glycerols Due to Hydrolysis of Menhaden Oil

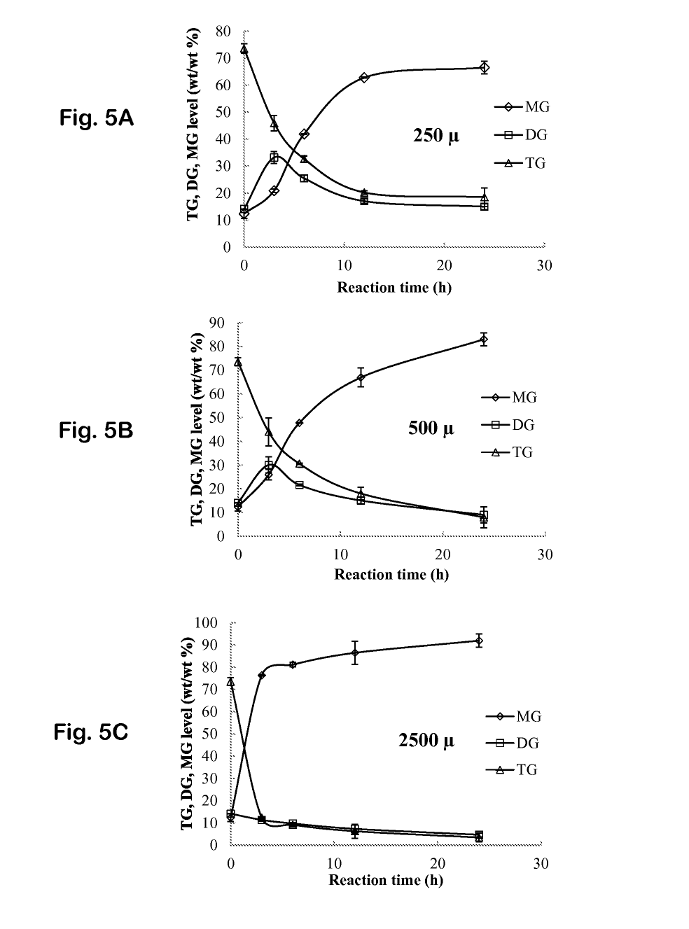

The changes in levels of monoglycerides (MG), diglycerides (DG), and triglycerides (TG) in the unhydrolyzed menhaden oil and final menhaden oils that were hydrolyzed at various time frames with 250, 500, and 2500 U of lipase are shown in FIGS. 5A, 5B, and 5C, respectively. The unhydrolyzed menhaden oil contained 73.53% TG, 14.13% DG, and 12.34% MG. TG levels were significantly less in all final hydrolyzed oil compared to the original menhaden oil. Using 250 U lipase, TG levels were significantly reduced from 73.53% to 45.92% after 3 h, and finally decreased to 18.50% after 24 h. DG levels significantly increased from 14.13% to 33.18% after 3 h of hydrolysis with 250 U lipase, and then gradually decreased to 14.99% after 24 h of hydrolysis. MG increased throughout the hydrolysis time, which resulted in a big increase in the MG level from 12.34% to 66.51% after 24 h.

At 500 U lipase, as shown in FIG. 5B, a similar trend was observed as at 250 U. The only main difference was that the TG and DG levels both showed greater overall decreases (TG: from 73.53% to 7.97% at 24 h, and DG: from 14.13% to 9.03% at 24 h). MG levels increased more than at 250 U (from 12.34% to 83.00% at 24 h). As shown in FIG. 5C, a much steeper change on the TG, DG, and MG levels occurred using 2500 U lipase. After 3 h of hydrolysis, the TG level decreased from 73.53% to 12.32% and gradually decreased to 3.43% after 24 h. DG level had a constant decrease with reaction time and dropped to 4.66% after 24 h. MG level was elevated dramatically from 12.34% to 76.34% after 3 h hydrolysis, and eventually increased to 91.90% after 24 h (FIG. 5C).

### Example 5

### Changes in Glycerols Due to Hydrolysis of Salmon Oil

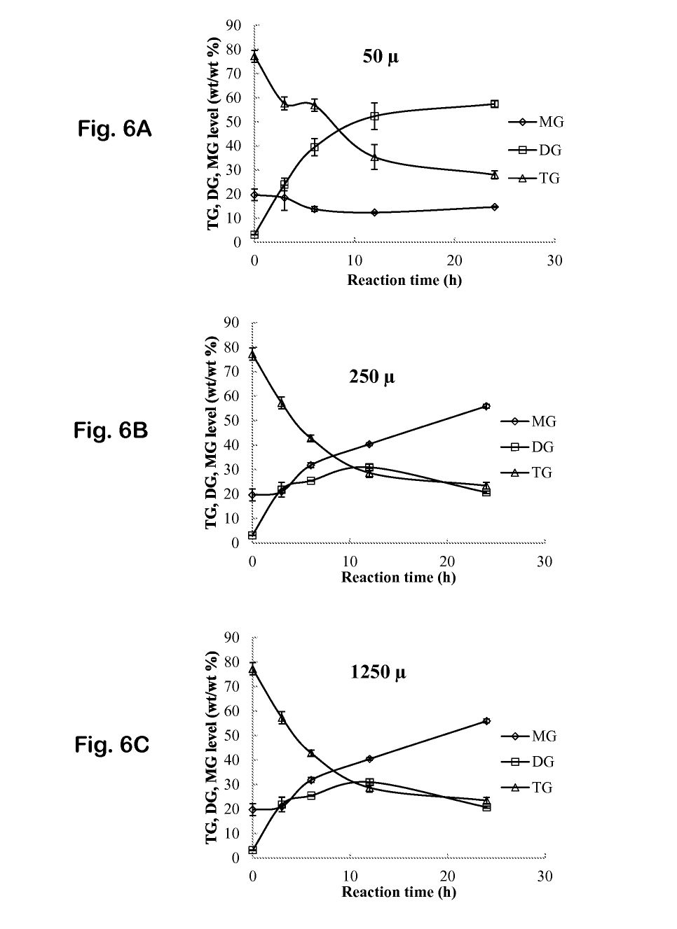

The changes in levels of monoglycerides (MG), diglycerides (DG), and triglycerides (TG) in the unhydrolyzed salmon oil and final salmon oils that were hydrolyzed at various time frames with 50, 250, and 1250 U lipase are shown in FIGS. 6A, 6B, and 6C, respectively.

The unhydrolyzed salmon oil contained 77.17% TG, 3.17% DG; and 19.53% MG. TG levels were significantly reduced from 77.17% to 57.57% with 50 U after 3 h and finally decreased to 27.99% after 24 h (FIG. 6A). DG levels significantly increased from 3.17% to 23.98% after 3 h of hydrolysis with 50 U lipase, and then gradually increased to 57.38% after 24 h of hydrolysis. This means most of TG was hydrolyzed to DG after 24 h. The lipase at 250 U showed a different trend than at 50 U. As shown in FIG. 6B, TG level decreased from 77.17% to 23.42%, but the DG levels were significantly increased to 30.95% at 12 h, but then dropped to 20.70% after 24 h of hydrolysis. Also, MG increased dramatically from 19.66% to 55.87% after 24 h hydrolysis.

There was a more obvious change in the TG, DG, and MG levels when using 1250 U of lipase, as shown in FIG. 6C. After 3 h of hydrolysis, the TG level decreased from 77.17% to 49.80%, and then decreased to 9.51% after 24 h. DG level decreased to 26.13% after 6 h of hydrolysis, and then dropped to 17.08% after 24 h. MG level was raised dramatically from 19.66% to 73.40% after 24 h of hydrolysis.

These results are similar to reported hydrolysis values for sardine oil. The tri-, di-, and monoglycerols in sardine oil changed from 86.20%, 13.40%, and 0.51% to 65.94%, 32.33%, and 2.08% after hydrolyzed with CR lipase for 9 h at 250 U and similar glycerol levels were found when 500 U of CR lipase were used (Okada et al., 2007). In that reported work, the lipase was inactivated by additional of ethanol, and KOH was added to neutralize the FFAs.

Results from EPA and DHA Enhancement:

Analysis of oils enhanced for EPA and DHA indicated that the highest degree of hydrolysis (DH) of menhaden and salmon oils (81.64% and 81.47%, respectively) were obtained by 2500 U and 1250 U lipase treatment for 24 h, respectively. Menhaden oil treated with 2500 U lipase for 6 and 12 h had the most total EPA and DHA fractions (39.54% and 39.95%, respectively) in the final oil, while the highest total EPA and DHA fractions (32.12%) in the salmon oil were found in the oil hydrolyzed with 1250 U lipase for 6 h. The unhydrolyzed menhaden source oil contained 73.53% TG, 14.13% DG, and 12.34% MG. After treated with 2500 U lipase for 24 h, the TG and DG levels of menhaden oil decreased to 3.43% and 4.66%, while MG level increased to 91.90%. The unhydrolyzed salmon source oil contained 77.17% TG, 3.17% DG, and 19.53% MG. After being hydrolyzed with 1250 U of lipase for 24 h, the TG, DG, and MG levels changed to 9.97%, 17.03%, and 72.87%. As expected, in both cases the levels of monoglycerols increased, while those of di- and tri-glycerols dropped.

We developed a method that optimized the lipase treatment for enrichment of EPA and DHA concentrations in menhaden and salmon oils. After the EPA and DHA enrichment, PV, FFA, and TBARs values of menhaden oil (enriched MO or MOE) all decreased from the original source oil: PV, from 11.06±0.75 meq/kg oil to 1.49±0.13 meq/kg oil; FFA, from 2.66±0.07% to 0.67±0.06%; and TBARs, from 0.89±0.01 mmol/kg to 0.56±0.01 mmol/kg. Similar tendencies were observed for salmon oil, with PV decreasing from 14.71±1.26 meq/kg oil to 2.33±0.16 meq/kg oil; FFA decreasing from 3.14±0.07% to 0.66±0.05%; and TBARs decreasing from 1.26±0.02 mmol/kg to 0.62±0.03 mmol/kg. This study developed a novel enzymatic method, optionally used in combination with the adsorption technology to purify the oil enriched for EPA and DHA without organic solvent and chemicals, and without the need for thermal inactivation of enzyme. We have shown that activated surface materials can be used to purify the oils.

### Example 6

### Purification Method with Activated Surface Materials and Analysis Methods

Activated Surface Materials.

The oils obtained from menhaden and salmon as discussed in Example 1 were used to analyze various purification methods using activated surface materials. The oils used below had not been treated with lipase. Shrimp chitosan was obtained from Green Pastures Products Inc. (O'Neill, Nebr.). Activated alumina was obtained from Zapp's Potato Chips Inc. (Gramercy, La.), and activated earth was obtained from the BASF Chemicals Division (Geismar, La.).

Purification Method.

This adsorption purification was conducted in glass containers. Thirty grams of the enhanced MO/SO was placed into each glass container with 1.5 g of an adsorbent or a combination of adsorbents (chitosan, activated earth or activated alumina) added. The adsorption reaction was carried out with constant agitation using a magnetic stirrer at room temperature, about 22±1° C. Experiments were repeated three times.

Five different batch adsorption processes were used to purify MO/SO: (1) process #1 involved purification using 5% (wt/wt of oil) chitosan (MCH/SCH); (2) process #2 involved purification by 5% (wt/wt of oil) activated earth (MAE/SAE); (3) process #3 involved purification by 5% (wt/wt of oil) activated alumina (MAA/SAA); (4) process #4 involved combined purification processes of 5% (wt/wt of oil) chitosan, 1.5% chitosan plus 3.5% activated earth, and 5% activated alumina (M4/S4) in 3 separate steps; and (5) process #5 involved combined purification processes of 5% (wt/wt of oil) chitosan, 4% chitosan plus 1% activated earth, and 5% activated alumina (M5/S5) in 3 steps.

Peroxide Values and Free Fatty Acids in Unrefined Fish Oils.

PV of the fish oils was determined by a titration method according to AOAC 965.33 (1999). The results were expressed in terms of milliequivalent peroxides per Kg of oil (meq/Kg). FFA content of the unrefined oil was determined using a titration method (AOCS Ca 5a-40, 1998), and the percentage of FFA was expressed as oleic acid equivalents.

Density, Specific Gravity, Water Activity, and Moisture Content of Oils.

Bulk density of the oils was determined in triplicate using a 25 mL glass-measuring cylinder at 25° C. The sample was filled to 25 mL, the weight to volume ratio determined, and bulk density values reported as g/mL. Specific gravity of the unrefined fish oils was determined in triplicate using a 25 mL glass-measuring cylinder. The net weight of the oil (g) was divided by the net weight of water (g) at 25° C. to obtain the specific gravity. A calibrated Rotronic water activity meter (AwQUICK, Rotronic Instrument Corp., Huntington, N.Y.) was used to measure the water activity of the unrefined oils at 25° C. The moisture content was measured according to the Karl Fischer titration method using a Mitsubishi Karl Fischer Moisturemeter (Mitsubishi Chemical Analytech Co., Ltd., Japan).

Iodine Value.

Iodine value, a measure of the unsaturation of the oils, was measured following the AOCS official method Cd 1-25 (1998). It was expressed in terms of number of centigrams of iodine adsorbed per gram of sample (% iodine adsorbed). All of the analyses were repeated three times.

Fatty Acid Profile and Mineral Concentrations Analyses.

Fatty acid composition of oil samples was determined at the USDA-ARS Laboratory, University of Alaska Fairbanks, Ak. Fatty acid methyl esters (FAMEs) were prepared using a previously described method (Maxwell et al., 1983), and as described above in Example 1.

Mineral content of oil samples was determined according to AOCS Ca17-01 and AOCS Ca 20-99 (1998) and reported as ppm. The mineral profile analysis of the oil samples was carried out in triplicate by the acid digestion method involving microwave technology (CEM microwave, MDS-2000, CEM 3 5 Corp., Matthews, N.C., U.S.A.). A 0.5 g sample was placed in a vessel, and 6 mL HNO3 was added: The sealed vessel was heated until digestion was completed. The samples were cooled for 5 min. The inductively coupled argon plasma system (Model CIROS, SPECTRO Analytical Instruments, Kleve, Germany) was utilized to determine the mineral profile.

### Example 7

### Peroxide Value (PV), Free Fatty Acids (FFA), Moisture, and Iodine Value (IV) of Unrefined and Refined Fish Oil

Peroxide value (PV) is a good indicator of initial lipid oxidation. The initial PV of unpurified menhaden and salmon oil were 24.22±1.24 and 38.59±0.42 meq/kg of fish oil (see Table 3 and Table 4 below). The results show that the MAE and SAE, which are the menhaden and salmon oils purified by the activated earth, have the lowest peroxide values (13.36±1.82 meq/Kg and 20.63±0.45 meq/Kg) among these oils. These results indicate that activated earth can effectively adsorb primary oxidation compounds compared with chitosan and activated alumina from both menhaden and salmon oils. The adsorption principles of activated earth on the oxidization products have been reported to be related to hydrogen bonding, competition for adsorption sites; electrostatic field strength and intraparticles diffusion of molecules (Huang et al., 2010).

Free fatty acid content (FFA) is one of the most harmful impurities in fish oils, and lowering FFA is a very important goal in oil purification process. Activated alumina decreased the free fatty acids of the menhaden and salmon oils from 2.76±0.29% to 2.14±0.06% and from 2.40±0.05% to 1.75±0.03%, respectively, after 1 h of adsorption. Activated alumina is an amorphous aluminum oxide from aluminum trihydrate. The free fatty acids are adsorbed due to the combination between aluminum oxides and the free fatty acids. Neither chitosan nor activated earth was effective in reducing FFA from the SO, which is in agreement with published results (Huang et al., 2010). The products M4 and M5 had similar FFA contents compared with MAA, while S4 and S5 had similar FFA contents compared with SAA.

Chitosan was the most effective adsorbent in reducing the moisture content, which decreased the moisture content of the menhaden and salmon oils from 3560±0.42 ppm and 631.60±37.9 ppm to 591.45±0.5 ppm to 162.6±6.4 ppm, respectively. Chitosan is recognized as a hydrophilic compound, and when added to the fish oils, it adsorbs water rapidly. Activated earth and activated alumina can also remove the moisture because of their adsorption capacities, and both activated earth and activated alumina reduced the moisture to 495.55±10.25 ppm and 399.95±2.05 ppm, respectively. M4 and M5 had the lowest moisture contents (256.55±6.72 ppm and 271.37±16.38 ppm) among all the menhaden oil samples. Similarly, S4 and S5 had the lowest moisture contents (143.3±3.0 ppm and 141.3±3.3 ppm) among all the salmon oil samples.

Iodine value (IV), also called iodine adsorption value or iodine number or iodine index, measures the degree of unsaturation of the oil and is expressed in terms of the number of centigrams of iodine adsorbed per gram of sample. Iodine value is one of the standard parameters related to chemical composition and quality of oils and can be used to follow the change in the fatty acid profile during processing or storage of the oil. In this study, the IV of the unrefined and refined menhaden oils were around 179.71 to 181.18 cg I₂/g oil, while the IV of salmon oils ranged from 170 to 178 cg I₂/g oil, which is a little higher than reported values (147.8-170 cg I₂/g oil) for Atlantic salmon (S. salar) (Afseth et al., 2006). Statistically there were no differences among the fish oil samples for IV. This confirms that the adsorption process did not change the degree of unsaturation and composition of fatty acid profile on the fish oils.

TABLE 3
PV, FFA, moisture, and IV of the unrefined and refined menhaden oils

| Sam- | | | | |
|---|---|---|---|---|
| ple | PV (meq/kg) | FFA (%) | Moisture (ppm) | IV (cg I₂/g oil) |
| MO | 24.22 ± 1.24^{a} | 2.76 ± 0.29^{a} | 3560 ± 0.42^{a} | 180.40 ± 3.21^{a} |
| MCH | 17.29 ± 0.06^{b} | 2.82 ± 0.10^{a} | 591.45 ± 0.49^{d} | 179.98 ± 2.14^{a} |
| MAE | 13.36 ± 1.82^{c} | 2.74 ± 0.05^{a} | 897.9 ± 16.97^{c} | 180.37 ± 2.45^{a} |
| MAA | 22.58 ± 1.06^{a} | 2.14 ± 0.06^{b} | 986.25 ± 11.67^{b} | 181.18 ± 0.56^{a} |
| M4 | 15.94 ± 0.41^{bc} | 2.00 ± 0.09^{b} | 256.55 ± 6.72^{c} | 179.71 ± 4.01^{a} |
| M5 | 15.88 ± 0.50^{bc} | 2.04 ± 0.08^{b} | 271.37 ± 16.38^{c} | 180.70 ± 2.89^{a} |

In Table 3, values are means±SD of triplicate determinations. ^{abed} Means with the same letters in each column are not significantly different (P>0.05). MO=unrefined menhaden oil; MCH=process involved purification of MO by 5% (wt/wt of oil) chitosan for 1 h; MAE=process involved purification of MO by 5% (wt/wt of oil) activated earth for 1 h; MAA=process involved purification of MO by 5% (wt/wt of oil) activated alumina for 1 h; M4=process involved combined MO purification processes of 5% (wt/wt of oil) chitosan, 1.5% chitosan plus 3.5% activated earth, and 5% activated alumina for 1 h, respectively; M5=process involved combined UPO purification processes of 5% (wt/wt of oil) chitosan, 4% chitosan plus 1% activated earth, and 5% activated alumina for 1 h, respectively. PV=peroxide value; FFA=free fatty acid content; IV=iodine value.

TABLE 4
PV, FFA, moisture, and IV of the unrefined and refined salmon oils

| | PV (meq/kg) | FFA (%) | Moisture (ppm) | IV (cg I₂/g oil) |
|---|---|---|---|---|
| SO | 38.59 ± 0.42^{a} | 2.40 ± 0.05^{a} | 631.6 ± 37.90^{a} | 176.02 ± 4.21^{a} |
| SCH | 35.20 ± 0.20^{b} | 2.32 ± 0.07^{a} | 162.6 ± 6.36^{d} | 177.68 ± 1.63^{a} |
| SAE | 20.63 ± 0.45^{d} | 2.45 ± 0.05^{a} | 495.55 ± 10.25^{b} | 177.19 ± 5.32^{a} |
| SAA | 34.19 ± 1.02^{b} | 1.75 ± 0.03^{b} | 399.95 ± 2.05^{c} | 170.57 ± 4.09^{a} |
| S4 | 24.34 ± 0.17^{c} | 1.76 ± 0.02^{b} | 143.3 ± 2.97^{d} | 175.95 ± 0.57^{a} |
| S5 | 26.03 ± 1.48^{c} | 1.80 ± 0.14^{b} | 141.3 ± 3.25^{d} | 174.22 ± 10.43^{a} |

In Table 4, values are means±SD of triplicate determinations. ^{abed} Means with the same letters in each column are not significantly different (P>0.05). SO=unrefined salmon oil; SCH=process involved purification of SO by 5% (wt/wt of oil) chitosan for 1 h; SAE process involved purification of SO by 5% (wt/wt of oil) activated earth for 1 h; SAE=process involved purification of SO by 5% (wt/wt of oil) activated alumina for 1 h; S4=process involved combined SO purification processes of 5% (wt/wt of oil) chitosan, 1.5% chitosan plus 3.5% activated earth, and 5% activated alumina, for 1 h, respectively; S5=process involved combined UPO purification processes of 5% (wt/wt of oil) chitosan, 4% chitosan plus 1% activated earth, and 5% activated alumina, for 1 h, respectively. PV=peroxide value; FFA=free fatty acid content; IV=iodine value.

### Example 8

### Fatty Acid Methyl. Ester (FAME) Composition Analyses

Table 5 and Table 6 (shown below) show the compositions of the main fatty acids in the unrefined and refined menhaden and salmon oils. No differences were found among these fatty acids in the unrefined and refined menhaden and salmon oils. The processes were conducted at room temperature without heating and under anaerobic conditions (the air in the amber bottles was replaced by nitrogen). Thus oxidation of the unsaturated fatty acids was low to none. The EPA and DHA concentrations ranged between 16.86-17.07% and 6.38-6.86% for menhaden oils and 11.49-11.77% and 10.34-10.89% for salmon oils, respectively.

TABLE 5
Fatty acid methyl ester (%) of the unrefined and refined menhaden oils

| Fatty Acid | MO | MCH | MAE | MAA | M4 | M5 |
|---|---|---|---|---|---|---|
| C14 | 10.63 ± 0.13^{a} | 10.98 ± 0.06^{a} | 10.54 ± 0.05^{a} | 10.67 ± 0.10^{a} | 10.63 ± 0.14 | 10.21 ± 0.10^{a} |
| C16 | 19.19 ± 0.05^{a} | 19.51 ± 0.06^{a} | 18.69 ± 0.15^{a} | 19.00 ± 0.11^{a} | 18.88 ± 0.12^{a} | 18.08 ± 0.06^{a} |
| C16:1n7 | 11.21 ± 0.15^{a} | 11.79 ± 0.17^{a} | 11.31 ± 0.06^{a} | 11.52 ± 0.04^{a} | 11.42 ± 0.03^{a} | 11.24 ± 0.06^{a} |
| C18:1n9 | 8.00 ± 0.07^{a} | 8.42 ± 0.08^{a} | 8.07 ± 0.03^{a} | 8.10 ± 0.06^{a} | 8.15 ± 0.05^{a} | 7.73 ± 0.02^{a} |
| C20:5n3 | 16.86 ± 0.18^{a} | 16.76 ± 0.14^{a} | 17.02 ± 0.17^{a} | 17.07 ± 0.15^{a} | 16.89 ± 0.07^{a} | 16.87 ± 0.18^{a} |
| C22:6n3 | 6.53 ± 0.03^{a} | 6.86 ± 0.09^{a} | 6.61 ± 0.09^{a} | 6.66 ± 0.06^{a} | 6.64 ± 0.13^{a} | 6.38 ± 0.02^{a} |

Values are means ± SD of triplicate determinations. Fatty acids less than 5% are not reported.
^{a}Means with the same letters in each column are not significantly different (P > 0.05).
See description of Table 3-for definitions of MO, MCH, MAE, MAA, M4, and M5.

TABLE 6
Fatty acid methyl ester (%) of the unrefined and refined salmon oils

| Fatty Acid | SO | SCH | SAE | SAA | S4 | S5 |
|---|---|---|---|---|---|---|
| C16 | 12.32 ± 0.03^{a} | 12.30 ± 0.31^{a} | 12.43 ± 0.19^{a} | 12.67 ± 0.06^{a} | 12.55 ± 0.23^{a} | 12.51 ± 0.19^{a} |
| C16:1n7 | 5.14 ± 0.05^{a} | 5.20 ± 0.12^{a} | 5.21 ± 0.07^{a} | 5.34 ± 0.06^{a} | 5.27 ± 0.14^{a} | 5.25 ± 0.06^{a} |
| C18:1n9 | 11.89 ± 0.03^{a} | 12.01 ± 0.23^{a} | 12.15 ± 0.16^{a} | 12.33 ± 0.04^{a} | 12.22 ± 0.15^{a} | 12.11 ± 0.18^{a} |
| C20:1n11 | 7.03 ± 0.05^{a} | 7.12 ± 0.09^{a} | 7.20 ± 0.10^{a} | 7.23 ± 0.08^{a} | 7.24 ± 0.13^{a} | 7.16 ± 0.12^{a} |
| C20:5n3 | 11.49 ± 0.07^{a} | 11.64 ± 0.19^{a} | 11.76 ± 0.22^{a} | 11.76 ± 0.09^{a} | 11.77 ± 0.15^{a} | 11.71 ± 0.15^{a} |
| C22:1n11 | 11.40 ± 0.14^{a} | 11.58 ± 0.16^{a} | 11.65 ± 0.20^{a} | 11.68 ± 0.13^{a} | 11.67 ± 0.23^{a} | 11.59 ± 0.13^{a} |
| C22:6n3 | 10.89 ± 0.15^{a} | 11.08 ± 0.12^{a} | 11.16 ± 0.28^{a} | 11.34 ± 0.39^{a} | 11.13 ± 0.52^{a} | 11.11 ± 0.12^{a} |

Values are means ± SD of triplicate determinations. Fatty acids less than 5% are not reported.
^{a}Means with the same letters in each column are not significantly different (P > 0.05).
See description of Table 4 for definitions of SO, SCH, SAE, SAA, S4, and S5.

### Example 9

### Mineral Concentrations of the Unrefined and Refined Fish Oils

Table 7 and Table 8 list the mineral and heavy metal contents of unrefined and refined oil samples. B, Fe, Zn, Al, Ca, Mg, Na, and Ar were the most abundant minerals in unrefined menhaden and salmon oils. After the five adsorption processes, most of these minerals decreased in concentration. As compared with chitosan and activated alumina, activated earth (AE) was the most effective in reducing B, Fe, and Zn from the menhaden oil. AE decreased the iron contents from 19.15 ppm to 7.08 ppm, which is below the acceptable level (8 ppm) for iron (Bimbo, 1998). For the salmon oil, activated earth effectively removed the Fe, Zn, Ca, S, and Na. Activated alumina removed significant amounts of Ca and Mg from the menhaden fish oil. Chitosan had highest capacity for adsorbing K which decreased from 7.83 to 4.95 ppm. Chitosan effectively decreased the Al levels in both menhaden and salmon oils; in contrast, activated alumina and activated earth increased the Al levels in the oil samples. This was probably from the alumina in the adsorbents. Activated earth has a porous aluminum/silicate composition with a pore diameter of 50,000 Angstroms. The difference in adsorption capacity of these minerals is related to the physical structure of these adsorbents, because the adsorbate interaction potential largely depends on the pore size and geometry of the adsorbents (Yang, 2003). MO and SO contained high contents of P (45.36 ppm and 38.60 ppm), which was reduced to 15.65 ppm and 17.88 ppm by activated earth. The phosphorus present in fish oils may be attributed to the phospholipids in the oil which is the main components of the gum presents in oils. Another complex that phosphorus can form is calcium-phosphate complexes (Young, 1986). Even though all the three adsorbents were effective in reducing minerals or heavy metals from menhaden and salmon oils, there were still considerable amounts of minerals left in the oils after the adsorption processes. The neutralization process has been reported to reduce most of the minerals (Ca, Fe, Mg, P, Na, Ar) in salmon oils to trace amounts (Huang et al., 2010). This could be caused by the washing step during the neutralization process which removes the water soluble impurities, especially phospholipids from the raw oil and most of minerals and heavy metals precipitated with the soap as saponification occurred during neutralization. The problem with the neutralization method is the increase of Na because of the addition of NaOH solution (Huang et al., 2010).

TABLE 7
Minerals and heavy metal concentration (ppm) of unrefined and refined menhaden oils

| | MO | MCH | MAE | MAA | M4 | M5 |
|---|---|---|---|---|---|---|
| Boron | 30.6 ± 1.13^{a} | 25.75 ± 1.90^{c} | 24.8 ± 0.42^{c} | 28.31 ± 1.38^{b} | 19.75 ± 1.21^{d} | 22.55 ± 1.34^{cd} |
| Copper | <1.2 | <1.2 | <1.2 | <1.2 | <1.2 | <1.2 |
| Iron | 19.15 ± 0.78^{a} | 10.43 ± 1.24^{c} | 7.08 ± 0.59^{d} | 15.4 ± 0.42^{b} | 8.07 ± 0.64^{d} | 10.3 ± 0.28^{c} |
| Manganese | <1 | <1 | <1 | <1 | <1 | <1 |
| Zinc | 4.50 ± 0.32^{a} | 4.06 ± 0.65^{b} | 1.08 ± 0.52^{d} | 3.48 ± 0.36^{c} | 0.99 ± 0.19^{d} | 1.06 ± 0.07^{d} |
| Aluminum | 5.34 ± 1.27^{b} | 3.83 ± 0.78^{c} | 6.06 ± 0.78^{a} | 6.94 ± 0.72^{a} | 4.59 ± 1.82^{b} | 4.73 ± 0.23^{bc} |
| Barium | <0.2 | <0.2 | <0.2 | <0.2 | <0.2 | <0.2 |
| Cadmium | <0.2 | <0.2 | <0.2 | <0.2 | <0.2 | <0.2 |
| Chromium | 1.80 ± 0.08^{a} | 1.24 ± 0.17^{b} | 0.89 ± 0.13^{c} | 1.31 ± 0.56^{b} | 1.19 ± 0.37^{b} | 1.26 ± 0.19^{b} |
| Calcium | 37.75 ± 1.48^{a} | 33.05 ± 1.34^{a} | 23.56 ± 1.36^{b} | 19.7 ± 2.40^{c} | 17.65 ± 1.03^{c} | 17.6 ± 1.27^{c} |
| Cobalt | <0.2 | <0.2 | <0.2 | <0.2 | <0.2 | <0.2 |
| Magnesium | 8.86 ± 0.21^{a} | 7.67 ± 0.71^{a} | 5.11 ± 0.06^{b} | 4.58 ± 0.23^{b} | 3.16 ± 0.25^{c} | 3.88 ± 0.03^{c} |
| Lead | <0.2 | <0.2 | <0.2 | <0.2 | <0.2 | <0.2 |
| Molybdenum | <0.8 | <0.8 | <0.8 | <0.8 | <0.8 | <0.8 |
| Phosphorus | 45.36 ± 1.36^{a} | 20.34 ± 1.22^{b} | 15.65 ± 0.38^{c} | 18.34 ± 2.65^{bc} | 17.56 ± 1.78^{bc} | 17.41 ± 0.89^{bc} |
| Potassium | 7.83 ± 0.46^{a} | 4.95 ± 0.92^{c} | 6.06 ± 0.48^{b} | 6.05 ± 1.06^{b} | 4.78 ± 0.28^{c} | 4.81 ± 0.02^{c} |
| Nickel | <0.4 | <0.4 | <0.4 | <0.4 | <0.4 | <0.4 |
| Selenium | <14 | <14 | <14 | <14 | <14 | <14 |
| Arsenic | 18.55 ± 1.62^{a} | 16.25 ± 0.92^{a} | <4 | <4 | <4 | <4 |
| Sodium | 36.15 ± 1.20^{a} | 21.9 ± 0.57^{b} | 22.2 ± 1.75^{b} | 20.6 ± 1.53^{b} | 17.1 ± 1.41^{c} | 20.6 ± 0.28^{b} |

Values are means ± SD of duplicate determinations.
^{abcd}Means with the same letters in each column are not significantly different (P > 0.05).
See description of Table 3 for definitions of MO, MCH, MAE, MAA, M4, and M5.

TABLE 8
Minerals and heavy metal concentration (ppm) of unrefined and refined salmon oils

| | SO | SCH | SAE | SAA | S4 | S5 |
|---|---|---|---|---|---|---|
| Boron | 36.20 ± 1.70^{a} | 32.50 ± 1.98^{b} | 34.80 ± 1.84^{a} | 29.15 ± 1.16^{c} | 33.00 ± 2.21^{b} | 29.15 ± 1.31^{c} |
| Copper | <0.2 | <0.2 | <0.2 | <0.2 | <0.2 | <0.2 |
| Iron | 5.90 ± 0.24^{a} | 5.39 ± 1.56^{a} | 1.25 ± 0.04^{c} | 3.03 ± 0.30^{b} | 1.86 ± 0.15^{c} | 1.52 ± 0.02^{c} |
| Manganese | <1 | <1 | <1 | <1 | <1 | <1 |
| Zinc | 4.85 ± 0.64^{a} | 4.06 ± 2.65^{ab} | 1.73 ± 0.62^{b} | 3.48 ± 1.36^{ab} | 3.93 ± 0.38^{ab} | 3.69 ± 0.87^{ab} |
| Aluminum | 12.60 ± 1.27^{b} | 7.83 ± 3.78^{c} | 16.00 ± 0.94^{a} | 16.25 ± 1.48^{a} | 11.23 ± 0.82^{b} | 10.60 ± 0.71^{b} |
| Barium | <0.2 | <0.2 | <0.2 | <0.2 | <0.2 | <0.2 |
| Cadmium | <0.2 | <0.2 | <0.2 | <0.2 | <0.2 | <0.2 |
| Chromium | 1.47 ± 0.08^{a} | 1.24 ± 0.17^{a} | 1.20 ± 0.23^{a} | 1.28 ± 0.24^{a} | 1.19 ± 0.37^{a} | 1.15 ± 0.26^{a} |
| Calcium | 42.87 ± 0.58^{a} | 29.86 ± 2.25^{b} | 17.90 ± 2.26^{c} | 25.50 ± 3.11^{b} | 27.65 ± 4.03^{b} | 26.10 ± 6.93^{b} |
| Cobalt | <0.2 | <0.2 | <0.2 | <0.2 | <0.2 | <0.2 |
| Magnesium | 8.53 ± 0.49^{a} | 7.67 ± 0.71^{ab} | 7.30 ± 0.26^{ab} | 7.47 ± 0.36^{ab} | 5.16 ± 0.29^{b} | 4.42 ± 0.18^{c} |
| Lead | <1.2 | <1.2 | <1.2 | <1.2 | <1.2 | <1.2 |
| Molybdenum | <0.8 | <0.8 | <0.8 | <0.8 | <0.8 | <0.8 |
| Phosphorus | 38.60 ± 2.26^{a} | 25.75 ± 0.35^{b} | 17.88 ± 0.21^{d} | 25.65 ± 2.33^{b} | 21.50 ± 0.37^{c} | 22.70 ± 1.13^{bc} |
| Potassium | 26.18 ± 2.23^{a} | 8.37 ± 2.17^{bc} | 6.06 ± 0.48^{c} | 8.21 ± 1.70^{bc} | 4.70 ± 0.68^{c} | 5.25 ± 0.29^{c} |
| Nickel | <0.4 | <0.4 | <0.4 | <0.4 | <0.4 | <0.4 |
| Sulphur | 71.9 ± 0.70^{a} | 71.15 ± 3.04^{a} | 50.75 ± 0.64^{c} | 59.85 ± 0.07^{b} | 67.55 ± 10.82^{ab} | 50.65 ± 1.48^{c} |
| Selenium | <0.5 | <0.5 | <0.5 | <0.5 | <0.5 | <0.5 |
| Arsenic | 6.07 ± 1.10^{a} | 4.84 ± 0.35^{b} | <4 | <4 | <4 | <4 |
| Sodium | 59.60 ± 5.66^{a} | 45.30 ± 4.10^{b} | 19.40 ± 1.70^{d} | 25.30 ± 3.82^{cd} | 28.00 ± 3.11^{c} | 30.05 ± 1.63^{c} |

Values are means ± SD of duplicate determinations.
^{abcd}Means with the same letters in each column are not significantly different (P > 0.05).
See description of Table 4 for definitions of SO, SCH, SAE, SAA, S4, and S5.

Results of Oil Purification with Activated Surface Materials:

As indicated above, the enriched oils after hydrolysis could be further purified using activated surface materials. We have shown that activated earth, activated alumina, and chitosan can be used to adsorb the impurities from menhaden and salmon fish oils. Generally, using a single adsorbent was not effective in removing all the impurities because of the diversity of these impurities and the limitation of each adsorbent. We have shown that a combined adsorption (activated earth) and neutralization process to purify salmon oil was more effective in reducing FFA, peroxides, and moisture contents than either the adsorption or neutralization process alone (Huang et al., 2010). However, the neutralization process frequently caused a higher oil loss. We have now used a combined adsorption purification process using three different adsorbents (chitosan, activated earth and activated alumina). In various embodiments of the invention, one or more activated surface materials may be used depending on the main form of the impurities in the oils, singly or in combinations; e.g., any one two or all three of chitosan, activated alumina and activated earth may be used for activated surface-based purification.

The results indicated that activated earth was most effective on reducing the primary oxidation compounds from hydrolyzed menhaden and salmon oils as compared with chitosan and activated alumina. Activated alumina was very effective on removing free fatty acids (FFA) from both oils in contrast to chitosan or activated earth neither of which was effective in reducing FFA. Chitosan was the best adsorbent for reducing the moisture in the fish oils. No difference among the Iodine value (IV) and fatty acid methyl ester (FAMEs) of the unrefined and refined oils were observed, indicating that the adsorption process did not change the degree of unsaturation and composition of fatty acid profile on either fish oil. Comparing with chitosan and activated alumina, activated earth was most effective reducing the elements of B, Fe, and Zn from the menhaden oil. For the salmon oil, activated earth effectively removed the elements of Fe, Zn, Ca, S, and Na. Activated alumina was more effective in removing Ca and Mg from the menhaden fish oil. Chitosan had the highest capacity adsorbing K and reduced this element from 7.83 to 4.95 ppm in MO. MO and SO contain high contents of P (45.36 ppm and 38.60 ppm, respectively), and P was reduced to 15.65 ppm and 17.88 ppm, respectively, by activated earth. From this study, a batch adsorption process was developed for further purification of menhaden and salmon oils by reducing the peroxide value, FFAs, moisture, and heavy metal content, but retaining the desired DHA and EPA fatty acid compositions.

### REFERENCES

[1] Afseth K. N., Wolda P. J. Segtnana H. V. 2006. The potential of Raman spectroscopy for characterisation of the fatty acid unsaturation of salmon. Analytica Chimica Acta. 572 (1), 85-92.
[2] AOCS. 1998. Official Methods and Recommended Practices of the American Oil Chemists' Society. Champaign, Ill.: American Oil Chemists' Society; #Ca 5a-40; Cd 1-25; Ca 17-01; Ca 20-99.
[3] Association of Official Analytical Chemists (AOAC). 1999. Official methods of analysis, 16th edn. AOAC International, Arlington; #965.33.
[4] Benatti P., Peluso G., Nicolai R., Calvani M. 2004. Polyunsaturated fatty acids: biochemical, nutritional and epigenetic properties, Journal of the American College of Nutrition. 23.281-302.
[5] Bera D., Lahiri D., Nag A. 2004. Kinetic studies on bleaching of edible oil using charred sawdust as a new adsorbent. Journal of Food Engineering. 65.33-36.
[6] Bimbo A. P. and Cowther J. B. 1991. Fish oils: Processing beyond crude oil. Infofish Int. 6.20-25.
[7] Bottino N. R., Vandenburg G. A., Reiser R. 1967. Resistance of certain long-chain polyunsaturated fatty acids of fish oils to pancreatic lipase hydrolysis. Lipids. 2(6). 489-493.
[8] Breivik H., Haraldsson G. G., Kristinsson B. 1997. Preparation of highly purified concentrates of eicosapentaenoic acid and docosahexaenoic acid. Journal of the American Oil Chemists' Society. 74:1425-1429.
[9] Chapman D. M. 1994. Benefits and limitations of a novel chlorophyll adsorbent. J Am Oil Chem Soc 71 (4) 397-400.
[10] Chang S. S. 1967. Processing of fish oils. In Fish Oils, (M. E. Stansby, ed.), pp. 206-221, Chapman and Hall, New York.
[11] Du D., Sato M., Mori M., Park E. Y. 2006. Repeated production of fatty acid methyl ester with activated bleaching earth in solvent-free system. Process Biochemistry. 41. 1849-1853.
[12] El-Boustani S., Colette C., Monnier L., Descomps B., Paulet C. A., and Mendey F. 1987. Enternal absorption in man of eicosapentaenoic acid in different chemical forms. Lipids. 22.711-714.
[13] Fleming H. L. 1991. Adsorption on aluminas—current applications. Adsorption and its applications in industry and environmental protection. 120.561-583.
[14] Fujita, T. and Makuta M., Method of Purifying Eicosapentaenoic Acid and Its Esters. U.S. Pat. No. 4,377,526, Mar. 22, 1983.
[15]. Hamazaki T., Hirai A., Terano T., Sajiki J., Kondo S., Fujita T., Tamura Y., and Kumagai A. 1982. Effects of orally administrated ethyl ester of eicosapentaenoic acid on pig-like substance production by rat aorta. Prostaglandins. 23.557-567.
[16] Horrocks L. A. and Yeo Y. K. 1999. Health benefits of docosahexaenoic acid (DHA). Pharmacological Research. 40.211-225.
[17] Hoshino T., Yamane T., and Shimuzu S. 1990. Selective hydrolysis of fish oil by lipase to concentrate ω3 polyunsaturated fatty acids. Agric. Biol. Chem. 54. 1459-1467.
[18] Huang J. and Sathivel S. 2010. Purifying salmon oil using adsorption, neutralization, and a combined neutralization and adsorption process. Journal of Food Engineering, 96, 51-58.
[19] Hui Y. H. 1996. Banley's Industrial Oil and Fat Products. 5th Edn. John Willey and Sons Inc USA. 4.192-196.
[20] Hymore F. K. 1996. Effects of some additives on the performance of acid activated clays in the bleaching of palm oil. Applied Clay Science. 10.379-385.
[21] Jaeger K. E. and Reetz M. T. 1998. Microbial lipases form versatile tools for biotechnology. Trends in Biotechnology. 16.396-403.
[22] Lara A. V., Park E. Y. 2004. Potential application of waste activated bleaching earth on the production of fatty acid alkyl esters using Candida Cylindracea lipase in organic solvent system. Enzyme and Microbial Technology. 34.270-277.
[23] Lawson L. D. and Hughes B. G. 1988. Human Absorption of Fish Oil Fatty Acids as Triacylglycerols, Free Fatty Acids or Ethyl Esters. Biochem. Biophys. Res. Comm. 152.328-335.
[24] Linder M., Fanni J., and Parmentier M. 2005. Proteolytic extraction of salmon oil and PUFA concentration by lipases. Marine biotechnology. 15.70-76.
[25] Liu S. C., Zhang C. H., Hong P. Z., Ji H. W. 2007. Lipase-catalysed acylglycerol synthesis of glycerol and n-3 PUFA from tuna oil: Optimisation of process parameters. Food Chemistry. 103(3): 1009-1015.
[26] Maehr H., Zenchoff G., and Coffen D. L. 1994. Enzymic enhancement of n-3 fatty acid content in fish oils. Journal of the American Oil Chemists' Society. 71.463-467.
[27] Marldey K. S. 1964. Fatty Acids, Their Chemistry, Properties, Production, and Uses, Markley, K S (ed.), 2nd edition, Part 3, pp. 1983-2123, New York: Interscience Publishers. Technique of separation. A. Distillation, salt solubility, low-temperature crystallization.
[28] Matouba E., Mabiala B., Nzikou J. M., Linder M., Fanni J., Desobry S, and Parmentier M. 2008. Rheological and thermal properties of salmon oil after Aspergillus oryzae lipolysis. Journal of Engineering and Applied Sciences. 3(8). 656-663.
[29] Maxwell R J, Manner W N. 1983. Systematic protocol for the accumulation of fatty-acid data from multiple tissue samples—tissue handling, lipid extraction and class separation, and capillary Gas-Chromatographic analysis. Lipids 18(7):453-459.
[30] Mucha M., Ludwiczak S., Kawinska A. 2005. Kinetics of water sorption by chitosan and its blends with poly(vinyl alcohol). Carbohydrate Polymers 62(1): 42-49.
[31] Mukherjee K. D., Kiewitt I., Hills M. J. 1993. Substrate specificities of lipases in view of kinetic resolution of unsaturated fatty acids. Applied Microbiology and Biotechnology. 40.489-493.
[32] Okada T. and Morrissey T. M. 2007. Production of n-3 polyunsaturated fatty acid concentrate from sardine oil by lipase-catalyzed hydrolysis. Food Chemistry. 103. 1411-1419.
[33] Proctor A. 1996. Novel adsorbents for edible oil processing. Institute of Food Technologists (1996 annual meeting).
[34] Robles M. A., Grima E. M., Gimenez G. A., and Gonzalez M. J. I. 1998. Downstream processing of algal polyunsaturated fatty acids. Biotechnology Advances. 16 (3). 517-580.
[35] Sathivel, S., Prinyawiwatkul, W., Negulescu, I. I., King, J. M. and Basnayake, B. F. A. 2003. Effects of purification process on the rheological properties of catfish oil. Journal of the American Oil Chemists' Society. 80.829-832.
[36] Sathivel S. 2005. Thermal and flow properties of oils from salmon head. J Am Oil Chem Soc 2: 147-151.
[37] Sathivel S, Huang J, Prinyawiwatkul W. 2008a. Thermal Properties and Applications of the Arrhenius Equation for Evaluating Viscosity and Oxidation Rates of Unrefined Pollock Oil. J Food Eng 84: 187-193.
[38] Sathivel S, Prinyawiwatkul W, Negulescu I I, King J M. 2008b. Determination of Melting Points, Specific Heat Capacity and Enthalpy of Catfish Visceral Oil during the Purification Process. J Am Oil Chem Soc 85: 291-296.
[39] Shahidi F, Wanasundara P K J P D, Wansundara U N. 1997. Changes in edible fats and oils during processing. J Food Lipids 4: 199-231.
[40] Shimada Y., Maruyama K., Okazaki S., Nakamura M., Sugihara A., and Tominagal Y. 1994. Enrichment of polyunsaturated fatty acids with Geotrichum candidum Lipase. Journal of the American Oil Chemists Society. 71.951-954.
[41] Shinowara G. Y. and Brown J. B. 1940. Studies on the chemistry of the fatty acids. VI. The application of crystallization methods to the isolation of arachidonic acid, with a comparison of the properties of this acid prepared by crystallization and by debromination. Observations on the structure of arachidonic acid. Journal of Biological Chemistry. 134.331-340.
[42] Song Y., Qiao W., Yoon S. 2005. Toluene adsorption on various activated carbons with different pore structures. New Carbon Materials. 20.294-298.
[43] Sun T., Pigott G. M., and Herwig R. P. 2002. Lipase-assisted concentration of n-3 polyunsaturated fatty acids from viscera of farmed Atlantic salmon (Salmo salar L.). Journal of Food Science. 67 (1). 130-136.
[44] Tanaka Y., Hirano J., and Funada T. 1992. Concentration of docosahexaenoic acid in glyceride by hydrolysis of fish oil with Candida cylindracea lipase. Journal of the American Oil Chemists' Society 69. 1210-1214.
[45] Uauy R. and Valenzuela A. 2000. Marine oils: the health benefits of n-3 fatty acids. Nutrition. 6(7/8). 680-684.
[46] Wanasundara N. U. and Shahidi F. 1998. Lipase-assisted concentration of n-3 polyunsaturated fatty acids in acylglycerols from marine oils. Journal of American Oil Chemists' Society. 75 (8). 945-951.
[47] Yadwad V. B., Ward O. P., and Noronha L. C. 1991. Application of lipase to concentrate the docosahexaenoic acid (DHA) fraction of fish oil. Biotech. Bioeng. 38.956-959.
[48] Yang R. T. 2003. Basic considerations for sorbent design, Adsorbents Fundamentals and Applications, John Wiley & Sons Inc., Hoboken, N.J., USA, pp. 12-15.
[49] Yang L. Y., Kuksis A., and Myher J. J. 1989. Lumenal hydrolysis of menhaden and rapeseed Oil and their fatty acid methyl and ethyl esters in the rats. Biochem. Cell Biol. 67.192-204.
[50] Young F. V. K. 1986. The chemical and physical properties of crude fish oils for refiners and hydrogenators, Fish Oil Bulletin No. 18, Fish oil International Association of Fish Meal Manufacture, Herefordshire, UK, pp. 1-19.
[51] Young F. V. K., Poot C., Biernoth E., Krog N., Davidson N. G. J. and Gunstone F. D., 1994. Processing of oils and fats. In The Lipid Handbook. 2^{nd} Ed., (F. D. Gunstone, J. L. Harwood and F. B. Padley, eds.) pp. 249-318, Chapman & Hall, New York.

The complete disclosures of all references cited in this application are hereby incorporated by reference. Specifically incorporated by reference is the following: Huaixia Yin, “Purification of fish oils and production of protein powders with EPA and DHA enriched fish oils,” a dissertation submitted to the Graduate Faculty of Louisiana State University and Agricultural and Mechanical College in December 2011. In the event of an otherwise irreconcilable conflict, however, the present specification shall control.

What is claimed:

1. An enzymatic method for enhancing the percentage of eicosapentaenoic acid (EPA) and docosahexaenoic acid (DHA) from a source oil by hydrolysis using the enzyme lipase, said method comprising the following steps:
(a) mixing the source oil comprising EPA and DHA with a particulate lipase;
(b) separating the mixture into three hydrophobicity layers by centrifugation; and
(c) collecting the top hydrophobicity layer with the hydrolyzed oil with the increased concentration of DHA and EPA;
whereby the enzyme lipase is not deactivated prior to step (b) and the particulate lipase and other aqueous impurities are not found in the top hydrophobicity layer.

2. The method of claim 1, wherein the source oil is a fish oil.

3. The method of claim 2, wherein the source fish oil is from the group of fish consisting of menhaden, salmon oil, or sardine.

4. The method of claim 1, wherein the source oil is menhaden oil.

5. The method of claim 1, wherein the source oil is salmon oil.

6. The method of claim 1, wherein the source oil is from a marine mammal.

7. The method of claim 1, wherein the lipase is produced by an organism selected from the group consisting of: Candida rugosa, Pseudomonas cepacia, Candida cylindracea, Rhizopus niveus, Chromobacterium viscosum, Geotrichum candidum, Pseudomonas sp., and Aspergillus oryzae.

8. The method of claim 8, wherein the lipase is produced by Candida rugosa.

9. The method of claim 1, wherein the mixture of step (a) is not neutralized by adding alkali to the mixture.

10. The method of claim 1, wherein the mixture of step (a) is placed under a nitrogen atomsphere.

11. The method of claim 1, further comprising purifying the collected oil of step (c) by removing the free fatty acids, said purification method comprising the following steps:
(a) mixing the collected oil with an activated surface material; and
(b) removing the activated surface material from the mixture; and
(c) collecting the purified oil remaining;
whereby the purified oil with an enhanced percentage of EPA and DHA has less free fatty acids that the collected oil.

12. The method of claim 11, wherein the activated surface material is selected from the group consisting of activated alumina, activated earth, and chitosan.

13. The method of claim 11, wherein the activated surface material is activated alumina.

14. The method of claim 11, wherein said purification method repeats steps (a) through (c) one or more times.

15. The method of claim 11, wherein the removing step is by separating the collected oil and activated surface material by centrifugation, and collecting the oil layer.

16. A method for enhancing the percentage of at least one of eicosapentaenoic acid (EPA) and/or docosahexaenoic acid (DHA) in fish oil using the enzyme lipase without the need for enzyme inactivation by either added chemicals or increased temperature, said method comprising:
(a) mixing the fish oil with particulate Candida rugosa lipase under a nitrogen atomsphere;
(b) separating the mixture into hydrophobicity layers by centrifugation;
(c) collecting the upper oil layer with enhanced amounts of EPA and DHA and fatty acids after centrifugation;
(d) mixing the collected oil with activated alumina to purify the oil by removing at least some free fatty acids; and
(e) separating the activated alumina from the purified oil; and
(f) collecting the purified oil.

17. The method of claim 16, wherein steps (d) through (f) are repeated one or more times.

18. The method of claim 16, wherein the fish oil is from the group of fish consisting of menhaden, salmon oil, or sardine.

19. The method of claim 16, wherein the source oil is menhaden oil.

20. The method of claim 16, wherein the source oil is salmon oil.
